(19) United States
(12) Patent Application Publication
(10) Pub. No.: US 2026/0077072 A1
(43) Pub. Date: Mar. 19, 2026

(54) METHODS AND COMPOSITIONS FOR IMAGING MALIGNANCIES IN THE BONE MARROW

(71) Applicant: Washington University, St. Louis, MO (US)

(72) Inventors: Monica SHOKEEN, St. Louis, MO (US); Samuel ACHILEFU, St. Louis, MO (US)

(21) Appl. No.: 18/993,779

(22) PCT Filed: Jul. 13, 2023
(86) PCT No.: PCT/US2023/070161

§ 371 Date: Jan. 13, 2025

Related U.S. Application Data
(60) Provisional application No. 63/388,877, filed on Jul. 13, 2022.

(51) Int. Cl. A61K 51/08; A61P 35/00; C07B 59/00; G06T 7/00

(52) U.S. Cl. CPC A61K 51/088; A61P 35/00; C07B 59/008; G06T 7/0012; A61K 2123/00; G06T 2207/30008

(57) ABSTRACT

Compositions, methods of synthesis, and methods of use for an imaging agent that preferentially binds to activated VLA4, including, but not limited to a peptide or peptidomimetic ligand radiolabeled for use in PET imaging to identify bone marrow malignancies, help select treatments for bone marrow malignancies, and monitor relapse in bone marrow malignancy patients after therapy are disclosed.

### CROSS-REFERENCE TO RELATED APPLICATIONS

This application claims priority from U.S. Provisional Application Ser. No. 63/388,877 filed on Jul. 13, 2022, which is incorporated herein by reference in its entirety.

### STATEMENT REGARDING FEDERALLY SPONSORED RESEARCH OR DEVELOPMENT

This invention was made with government support under CA176221 awarded by the National Institutes of Health. The government has certain rights in the invention.

### MATERIAL INCORPORATED-BY-REFERENCE

Not applicable.

### FIELD OF THE INVENTION

The present disclosure generally relates to methods and compositions for imaging malignancies in the bone marrow.

### BACKGROUND OF THE INVENTION

Recent advances in the development of molecularly targeted radiopharmaceuticals have made possible the realization of precision medicine. In oncology, positron emission tomography (PET) performed with molecularly targeted radiolabeled vectors is an essential modality for patient management and improving outcomes. Development and utilization of diverse disease-specific small molecule and antibody-based probes are transforming our understanding of oncogenesis. Furthermore, there is increasing evidence of superior accuracy when nuclear imaging is synergized with liquid biopsies.

PET imaging performed with the metabolic radiopharmaceutical, [¹⁸F]fluorodeoxyglucose (FDG), has been the leading nuclear medicine tracer as demonstrated by its wide availability and frequent use. Of note, as compared to solid tumors, in hematological malignancies, FDG-PET/CT remains the mainstay for imaging of extramedullary infiltration, relapse, and assessment of inflammatory activity in leukemia as well as in FDG-avid lymphoma. Multiple myeloma is the second most common hematological cancer that causes debilitating end-organ symptoms and remains incurable. It is a disease of malignant plasma cells that originates in the bone marrow. Myeloma disorder is characterized by multiple precursor states: monoclonal gammopathy of undetermined significance and smoldering multiple myeloma. While the precursor states are not symptomatic, they are not benign either and present with a variable progression rate to overt myeloma. The unstable genome, inter-intra- and spatial tumoral heterogeneity, age, and immunosuppressive bone marrow microenvironment all contribute to the complexity and non-uniformity of myeloma pathogenesis. Consequently, the therapy options for myeloma encompass a combination of corticosteroids, immunomodulatory agents, proteasome inhibitors, immunotherapies, and bone marrow transplantation.

Interestingly, the pathological features of multiple myeloma include the presentation of both diffuse and punctate focal lesions in the bone marrow as well as in the skeleton and visceral organs. These disease features allow for a highly informative role of PET and functional magnetic resonance imaging (MRI) in the management of patients with multiple myeloma from the initial diagnosis to therapy monitoring. Detection of minimal residual disease, relapse, and therapy response is of utmost importance in multiple myeloma patients as well. Imaging plays a forefront role in this regard. Metabolic imaging with FDG-PET has been tremendously successful in multiple myeloma patients who present with FDG avid myeloma lesions. Despite its utility, however, a known limitation of FDG-PET in multiple myeloma is the inconsistent expression of glucose transporter, GLUT-1, and hexokinase-2 enzyme in myeloma cells. FDG uptake can additionally change during the course of disease progression and the following therapy. Collectively, the inflammatory tumor microenvironment in multiple myeloma significantly influences the FDG signal to background in the bone marrow, leading to either an over-estimation or under-estimation of disease burden. Therefore, the development of new molecularly-targeted tracers for imaging multiple myeloma that can overcome the limitations described above is the logical next step. It is encouraging to witness an exciting array of new tracers in multiple myeloma targeted toward metabolism and altered proteins such as CD38, CXCR4, and BCMA.

Myeloma cells thrive on pathogenic interactions with the cellular and non-cellular components of the bone marrow. One of the molecules that contributes significantly to the vicious cycle of the myeloma-bone marrow interaction is the integrin very late antigen-4 (VLA4). VLA4 is relatively overexpressed on multiple myeloma cells and is an established marker of cell adhesion mediated-drug resistance (CAM-DR).

### SUMMARY OF THE INVENTION

Among the various aspects of the present disclosure is the provision of methods and compositions for imaging malignancies in the bone marrow.

Briefly, therefore, the present disclosure is directed to methods and compositions to specifically target malignancies in the bone marrow.

In one aspect, compositions for an imaging agent that include a targeting ligand covalently attached to or complexed with a radionuclide binding structure are disclosed. The targeting ligand preferentially binds to activated VLA4, and the radionuclide binding structure is configured to bind or complex to a radionuclide. In some aspects, the targeting ligand is LLP2A or a modified LLP2A structure. In some embodiments, the radionuclide binding structure includes a chelator. In some embodiments, the chelator includes one of 1,4,8,11-tetraazacyclotetradecane-1-(methane phosphonic acid)-8-(methane carboxylic acid) (TE1A1P), dodecane tetraacetic acid (DOTA), diethylenetriaminepentaacetic acid (DTPA), or 1,4,8,11-tetraazacyclotetradecane-1-(methane phosphonic acid)-8-(methane carboxylic acid) (CB-TE1A1P). In some embodiments the imaging agent includes one of LLP2A-11-bis(carboxymethyl)-1,4,8,11-tetraazabicyclo[6.6.2]hexadecane (LLP2A-CB-TE2A), LLP2A-1,4,7,10-tetraazacyclododecane-N,N′,N″, N″-tetraacetic acid (LLP2A-DOTA), and LLP2A-DOTA-polyethylene glycol (LLP2A-DOTA-PEG). In some embodiments, the radionuclide includes a radiometal. In some embodiments, the radiometal includes one of ⁶⁴Cu, ¹¹¹In, or ⁶⁸Ga. In a particular embodiment, the imaging agent is [⁶⁴Cu]Cu-LLP2A. In some embodiments, the imaging agent further includes a linker covalently attached to or complexed with the targeting ligand and the radionuclide binding structure; the linker is configured to attach the targeting ligand to the radionuclide binding structure. In some aspects, the targeting ligand, the radionuclide binding structure, the linker, and any combination thereof are configured to modulate the activation of VLA4. In some embodiments, the imaging agent composition is a clinical dose. In some embodiments, a clinical dose of the composition includes about 15 μg to about 100 μg of a non-radioactive peptide portion that includes the targeting ligand, the radionuclide binding structure, the linker, and any combination thereof and from about 7 mCi to about 73 mCi of ⁶⁴Cu.

In various aspects, a method of synthesizing the imaging agent composition described above using good manufacturing practice is described.

In various aspects, a kit that includes the imaging agent composition described above is described.

In various aspects, a method for imaging malignant bone marrow in a subject is described that includes administering a clinical dose of the imaging agent composition described above, performing PET imaging on the subject, and spatially identifying lesions in the bone marrow based on the PET images In some embodiments, the PET imaging is performed from about one hour to about 6 hours after administering the clinical dose of the imaging agent composition. In some embodiments, the clinical dose of the imaging agent composition is administered by injection.

In various other aspects, a method is described for monitoring for relapse in malignant bone marrow in a subject that includes obtaining a baseline spatial map of lesions in the bone marrow of the subject after completion of a treatment and at least one follow-up map of lesions in the bone marrow of the subject at least once after obtaining the baseline spatial map. Each spatial map is obtained by administering a clinical dose of the imaging agent composition described above, performing PET imaging on the subject, and transforming the PET image into the spatial map of the lesions in the bone marrow. The method further includes comparing the at least one follow-up map to the at least one baseline spatial map and identifying a relapse if any of the at least follow-up maps include bone marrow lesions more extensive than the baseline map. In some embodiments, the PET imaging is performed from about one hour to about 6 hours after administering the clinical dose of the imaging agent composition. In some embodiments, the clinical dose of the imaging agent composition is administered by injection.

In various aspects, a method for selecting a treatment for malignant bone marrow in a subject is described that includes administering a clinical dose of the imaging agent described above, performing PET imaging on the subject, transforming the PET image into a spatial map of the lesions in the bone marrow, and selecting a treatment based on the spatial map of the lesions in the bone marrow. In some embodiments, the PET imaging is performed from about one hour to about 6 hours after administering the clinical dose of the imaging agent composition. In some embodiments, the clinical dose of the imaging agent composition is administered by injection. In some embodiments, the malignant bone marrow is associated with multiple myeloma.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1 is an image of the experimental setup in the chemistry hot cell for radiolabeling the precursor (LLP2A) with Copper-64.

FIG. 2A is a representative radio-HPLC chromatogram of ⁶⁴Cu-LLP2A showing radiochemical purity.

FIG. 2B is a table describing the pre- and post-release quality control specifications for the clinical-grade imaging agent.

FIG. 3A shows the percentage cell uptake of ⁶⁴Cu-LLP2A in 5TGM-GFP (murine myeloma) cells at 37° C. in the presence and absence of excess blocking dose of cold LLP2A peptide.

FIG. 3B is a table showing values of % blocking for each assay done during the radiolabeling validations and for clinical studies with study subjects.

FIG. 4A is a body weight vs. study day graph to evaluate the toxicity of ⁶⁴Cu-LLP2A in male CD-1 IGS mice.

FIG. 4B is a body weight vs. study day graph to evaluate the toxicity of ⁶⁴Cu-LLP2A in female CD-1 IGS mice.

FIG. 4C is a summary of the clinical observations determining the toxicity of Cu-LLP2A in male CD-1 IGS mice.

FIG. 4D is a summary of the clinical observations determining the toxicity of Cu-LLP2A in female CD-1 IGS mice.

FIG. 5A is an aggregated time-activity curve for the liver determined by calculating an organ time-integrated activity (TIA) on a per-patient basis by analytical integration of mono- or dual-exponent fits for the liver time-activity curves.

FIG. 5B is an aggregated time-activity curve for the kidney determined in the manner described in FIG. 5A.

FIG. 5C is an aggregated time-activity curve for the spleen determined in the manner described in FIG. 5A.

FIG. 5D is an aggregated time-activity curve for the blood pool determined in the manner described in FIG. 5A.

FIG. 5E is an aggregated time-activity curve for the bone marrow determined in the manner described in FIG. 5A.

FIG. 5F is a curve of aggregated excretion of the imaging agent in the urine over time.

FIG. 6 is a table of the organ radiation doses after injection of the imaging agent in nine human subjects.

FIG. 7 is a table of the organ residence times after injection of the imaging agent in nine human subjects.

FIG. 8 is a radio-HPLC chromatogram of the ⁶⁴Cu-LLP2A plasma sample fractions, 1 h post-injection of ⁶⁴Cu-LLP2A in healthy volunteers and myeloma patients. The elution time of intact peptide, ⁶⁴Cu-LLP2A is indicated by the yellow box whereas the elution time of free Copper-64 is indicated by the pink box.

FIG. 9A contains PET images from a multiple myeloma patient (left panels) and a healthy patient (right panels) after injection with the imaging agent that show enhanced uptake of the diseased patients.

FIG. 9B is a graph comparing ⁶⁴Cu-LLP2A standardized uptake values (SUVₘₐₓ) of the iliac bones in healthy and MM participants at an average of 240 min post-injection of the radiotracer. The mean and standard deviation for SUVₘₐₓ in healthy participants (n=5) was 12.05±2.0 while it was 25.62±9.38 for MM patients (n=3) (two-tailed; **P<0.03).

FIG. 10A shows ⁶⁴Cu-LLP2A PET/CT images acquired at 4h post-injection of the radiotracer using two different specific activities (14.36 MBq/nmol & 45.90 MBq/nmol) in healthy males and female mice with no tumor.

FIG. 10B shows ⁶⁴Cu-LLP2A PET/CT images acquired at 4h post-injection of the radiotracer using two different specific activities (14.36 MBq/nmol & 45.90 MBq/nmol) in female mice bearing 5TGM-GFP subcutaneous tumor.

FIG. 10C shows ⁶⁴Cu-LLP2A PET/CT images acquired at 4h post-injection of the radiotracer using two different specific activities (14.36 MBq/nmol & 45.90 MBq/nmol) in male (left images) and female (right images) mice bearing 5TGM-GFP systemic tumors.

FIG. 11A is a graph summarizing VLA4 expression from cells in the bone marrow of a multiple myeloma patient by flow cytometry.

FIG. 11B is a graph summarizing VLA4 expression from cells in the peripheral blood of a multiple myeloma patient by flow cytometry.

FIG. 12A is a flow cytometry gating strategy used to define eighteen hematopoietic cell subsets in a merged flow cytometry file of BM and/or peripheral blood (PB) mononuclear cells from healthy donors (UPNs 1954, 2055, 2140) and a patient (UPN99520, MMDM02) with MM at baseline and after disease progression four months later.

FIG. 12B is a t-SNE (t-distributed stochastic neighbor embedding) plot. tSNE projection of merged flow cytometry file showing eighteen hematopoietic populations based on defined cell surface markers described in FIG. 12A.

FIG. 13A shows the results of a flow cytometry analysis of LLP2A-Cy5 staining of human bone marrow and peripheral blood cell subsets. Heatmap showing the percentage of different cell subsets found within the bone marrow (BM) and/or peripheral blood (PB) of healthy donors (UPNs 1954, 2055, 2140) or a patient (UPN99520, MMDM02) with MM at baseline and after disease progression four months later.

FIG. 13B is a flow cytometry heatmap showing the mean fluorescence intensity of LLP2A-Cy5 staining of different cell subsets.

FIG. 14A is a plot of individual t-SNE representations from a merged flow cytometry file.

FIG. 14B is a quantification plot of hematopoietic cell subset distributions from the data in FIG. 14A; each defined population is shown as a percentage of the total CD45+ cells.

FIG. 15 is a set of flow cytometry heatmaps of parameters included in the t-SNE analysis.

FIG. 16 is a set of PET images that compares anterior maximum intensity projection images of ⁶⁴Cu-LLP2A-PET of a normal volunteer (MMDN05, upper panel) and a subject with multiple myeloma (MMDM03, lower panel) at similar time points after tracer injection. The best quality images were obtained between 1-5 h post-injection of the radiotracer. The later time points (˜24 h) exhibited relatively lower counts and high image noise.

FIG. 17 is a graph of MV SUV values in MM and healthy patients. BM SUV was measured as the average of lumbar vertebra 3,4 and 5 for most patients. For some patients, the marrow uptake was non-uniform in those regions, in which case marrow uptake in the lumbar 1 or 2, or thoracic 11-12 was used. Data were taken at the second imaging time point and ranged from 1 to 4 h post-injection. *Levene's test (equal variances not assumed), one-tailed, p=0.072 (Borderline statistically significant for n=9).

FIG. 18 is a set of PET images from a multiple myeloma patient. MM patient underwent PET imaging with ⁶⁴Cu-LLP2A and ¹⁸F-FDG. On PET/CT, an osteolytic lesion in the right iliac bone (arrows) of a MM patient had ¹⁸F-FDG uptake similar to background marrow. On PET/MRI, this same lesion (arrows) had ⁶⁴Cu-LLP2A uptake above the background marrow, corresponding to a fat-replacing lesion on fat-only Dixon images and a hyperintense lesion on In this lesion, the ⁶⁴Cu-LLP2A SUV-max was 29.5 with an SUL-peak (per PERCIST) of 18.7; in comparison, the ¹⁸F-FDG SUV-max was 2.9, with an SUL-peak (per PERCIST) of 2.1.

FIG. 19 is a table summarizing sequential HPLC measurements quantifying the stability of the Cu-CB-TE1A1P-PEG4-LLP2A V2 (Cu-LLP2A V2) tracer/imaging agent over time.

FIG. 20 is a graph summarizing the results of the Mean SUV Analysis of a toxicology study described in the Examples below.

FIG. 21 is a graph summarizing the results of the Max SUV Analysis of the toxicology study described above.

### DETAILED DESCRIPTION OF THE INVENTION

Among the various aspects of the present disclosure are the provision of VLA4-targeting PET imaging agent compositions, methods to synthesize the disclosed compositions, and methods to identify cancer lesions in the bone marrow using the disclosed compositions. The transcriptomic and biological effects of VLA4 modulation in myeloma cells were recently described, and the disclosed PET imaging probe in one aspect, ⁶⁴Cu-LLP2A, is specific for the activated conformation of VLA4.

The present teachings include compositions, methods of synthesis, and methods of use for an imaging agent that preferentially binds to activated VLA4, including but not limited to a peptide or peptidomimetic ligand radiolabeled for use in PET imaging to identify bone marrow malignancies, help select treatments for bone marrow malignancies, and monitor relapse in bone marrow malignancy patients after therapy. In some aspects, the disclosed imaging agent comprises the structure:

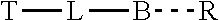

where T denotes a targeting ligand, L denotes a linker, B denotes a radionuclide binding structure, and R denotes a radionuclide used to radiolabel the imaging agent. In some aspects, the radionuclide is complexed to the remainder of the imaging agent by chelation, denoted above as a double dash (--). In some aspects, the structures are modifications of imaging agents described in De Nardo et al. J Nucl Med. 2009 April; 50 (4): 625-34, incorporated herein by reference in its entirety, which use LLP2A as T to target VLA4. By way of non-limiting example, the imaging agent is the VLA4-targeted clinical-grade imaging agent, [⁶⁴Cu]Cu-LLP2A.

In various aspects, the targeting ligand T comprises a peptide or peptidomimetic ligand configured to preferentially bind to VLA4. In some aspects, the targeting ligand T is further configured to modulate the binding affinity to activated VLA4 for optimized use in human subjects. Without being limited to any the VLA4 targeting molecule is thought to modulate the expression of the VLA4 pathway, imparting the high contrast performance achieved in vivo, including in human subjects, as demonstrated in the examples described herein.

In some aspects, the targeting ligand T comprises LLP2A (N-[[4-[[[(2-ethylphenyl)amino]carbonyl]amino]phenyl]acetyl]-Ne-6-[(2E)-1-oxo-3-(3-pyridinyl-2-propenyl)]-L-lysyl-L-2-aminohexanedioyl-(1-amino-1-cyclohexane) carboxamide (LLP2A) or modifications thereof. Without being limited to any particular theory, LLP2A has previously demonstrated the ability to detect tumors such as multiple myeloma but additionally accumulates in surrounding normal bone marrow tissue, leading to a significant background signal. In various aspects, the LLP2A structural framework may be modified by the inclusion of various atoms or other moieties strategically positioned to improve the tumor-to-background signal within a few hours post-injection. Non-limiting examples of atoms and other moieties suitable for modification of the LLP2A structural framework include atoms selected from fluorinated atoms, polyethylene glycols, amino acids, amino acid mimetics, organic molecules, phosphonates, sulfonates, carbohydrates, and polydentate functional groups. The modified LLP2A compounds are configured to bind to VLA-4 with high binding affinity, to clear rapidly from nontumor tissues, highlight the location of tumors, and/or determine the response of tumors to treatment.

In various aspects, the linker L may be any suitable linker moiety capable of binding together the targeting ligand T and the radionuclide binding structure B while also maintaining the targeting affinity of the targeting ligand T and the radionuclide binding properties of the radionuclide binding structure B. In some aspects, the linker L may include modifications configured to modulate the binding affinity of the imaging agent to VLA4 and or the expression of the VLA4 pathway, thereby optimizing the clinical performance of the imaging agent as described above.

In various aspects, the radionuclide binding structure B may be any suitable binding structure capable of binding or complexing to a radionuclide without limitation. Non-limiting examples of suitable radionuclide binding structures include chelators. Non-limiting examples of suitable chelators include chelators that coordinate metal cations such as 1,4,8,11-tetraazacyclotetradecane-1-(methane phosphonic acid)-8-(methane carboxylic acid) (TE1A1P), dodecane tetraacetic acid (DOTA), and diethylenetriaminepentaacetic acid (DTPA). In one exemplary embodiment, the radionuclide binding structure comprises a cross-bridged chelator 1,4,8,11-tetraazacyclotetradecane-1-(methane phosphonic acid)-8-(methane carboxylic acid) (CB-TE1A1P).

In other aspects, the non-radioactive peptide portion (T-L-B or T-B as described below) is radiolabeled with a radionuclide (R) to form the imaging agent. In various aspects, the radionuclide may be any suitable radionuclide known to function as a radiolabel in imaging agents without limitation. In some aspects, the radionuclide may be a radiometal. Non-limiting examples of suitable radiometals include ⁶⁴Cu, ¹¹¹In, and ⁶⁸Ga. The radionuclide may be selected based any one or more of at least several properties of the radionuclide including, but not limited to, the half-life of the radionuclide. Without being limited to any particular theory, the use of a radionuclide with a longer half-life extends the useable life of the imaging agent and facilitates the shipping of a targeting agent produced at one location to a different location for use in an imaging procedure. In one exemplary aspect, the radionuclide is ⁶⁴Cu. In another exemplary aspect, ⁶⁴Cu is coordinated to CB-TE1A1P to form the imaging agent.

In various other aspects, the targeting ligand is directly bound covalently to the radionuclide binding structure without a linker. In these other aspects, the imaging agent comprises the structure:

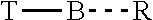

In some aspects, the imaging agent is configured to modulate the expression of an antigen in malignancies in the bone marrow including, but not limited to, VLA4.

Non-limiting examples of LLP2A modifications for use as imaging agents include LLP2A-11-bis(carboxymethyl)-1,4,8,11-tetraazabicyclo[6.6.2]hexadecane (LLP2A-CB-TE2A), LLP2A-1,4,7,10-tetraazacyclododecane-N,N′,N″, N′″-tetraacetic acid (LLP2A-DOTA), and LLP2A-DOTA-polyethylene glycol (LLP2A-DOTA-PEG). Without being limited to any particular theory, at least some LLP2A modifications are designed to modulate the binding affinity of the imaging agent to VLA4 in order to enhance the clinical performance of the imaging agent by providing maximum contrast and spatial imaging of bone marrow malignancies with high signal to noise characteristics.

Without being limited to any particular theory, the absolute amounts and the ratios of the molar masses of the T-L-B/T-B and R components of the disclosed compositions influence the in vivo and clinical performance of the imaging agent. In some aspects, the imaging agent contains a molar excess of the non-radioactive peptide portion (T-L-B or T-B as described above), resulting in low specific activity of the imaging agent. In various other aspects, the composition of the imaging agent comprises from about 15 μg to about 100 μg of the non-radioactive molecule (T-L-B) and from about 7 mCi to about 73 mCi of ⁶⁴Cu; compositions comprising these ranges of non-radioactive molecule and radionuclide have been proven safe and shown promise in identifying bone marrow malignancy in multiple myeloma patients in a first-in-human trial, as described in the examples herein.

The present teachings also include methods for the synthesis of the imaging agent, including but not limited to the production of good manufacturing practice (GMP) grade material for use in human subjects. In various aspects, a method for the production of a VLA4-targeted clinical-grade imaging agent, [⁶⁴Cu]Cu-LLP2A, under good manufacturing practice (GMP) conditions in a cyclotron facility for use in human subjects is disclosed herein. As described in the examples below, toxicity studies in rodents were conducted to test the safety of injecting up to 15 μg of the peptide dose in patients. In addition, the effects of doses up to 150-fold the 15 μg dose were evaluated in male and female mice. No adverse effects were noted and a dose of 15 μg was found to be None Observed Effect Level (NOEL).

In other examples described below, six healthy subjects and three subjects diagnosed with multiple myeloma were injected with the radioactive [⁶⁴Cu]Cu-LLP2A (7-11 mCi range). Blood was drawn from patients for evaluation of radioactive tracer metabolites. Due to the limitation of the sensitivity of detection resulting from the dilution over time, the metabolite study was performed to study stability up to 1h post-injection of the tracer in the human subjects. The tracer was found to be >95% stable, with free copper as the other major metabolite. The effective dose of this tracer was within the range reported for other copper radiopharmaceuticals (e.g., ⁶⁴Cu-SARTATE (0.0204 mGy/MBq), ⁶⁴Cu-ATSM (0.036 mSv/MBq), ⁶⁴Cu-DOTA-AE105 (0.0284 mSv/MBq) or ⁶⁴Cu-DOTATATE (0.0315 mSv/MBq) versus ⁶⁴Cu-LLP2A (0.036 mGy/MBq)). The organ with the highest dose was the spleen at a gender averaged value of 0.142 mSv/MBq, followed by the red marrow (0.104 mSv/MBq) and the bladder wall (0.094 mSv/MBq).

The pre-release acceptance criteria include factors including but not limited to pH, >90% radiochemical purity, >99% radionuclide purity, and negative endotoxin result. The post-release QC included the evaluation of the bioactivity of the radiopharmaceutical via a cell uptake assay in a VLA4-positive myeloma cell line 5TGM1, in the presence and absence of a blocking dose. Three multiple myeloma patients (1F and 2M) and six healthy subjects (3M and 3F) consented to take part in the first-in-human safety and dosimetry study. The tracer pharmacokinetics in humans closely followed the rodent data, with rapid washout from blood and clearance via kidneys and bladder. As expected, there was high uptake in the bone marrow. The residence time in the bone marrow was generally higher in females as compared to males and overall, higher in myeloma patients compared with healthy volunteers. The SUV analysis of the bone marrow in the right and left iliac bones showed significantly higher values in the multiple myeloma patients as compared to the normal subjects. The standard deviation was also higher in the myeloma patients' SUV data across all time points as compared to the SUVs of healthy subjects. The comparatively high variability of the SUV data of myeloma patients relative to healthy controls is indicative of the inhomogeneity/patchiness of the malignant bone marrow in the myeloma patients as compared to healthy marrow. This example, described in additional detail below, focused on the iliac bone SUVs (max, mean) and SUVs normalized to liver respectively.

In addition, as described in the examples herein, a comparison of image quality from early and late time points supports the selection of early time points, i.e., 4-6 h post-injection of the radiotracer as optimal. This is advantageous for the convenience of myeloma patients, as they prefer same-day relatively fast imaging due to the morbidity associated with the myeloma disease burden.

The present disclosure is based, at least in part, on the discovery that imaging agent provides contrast to malignancies in bone marrow in human subjects. As described herein, a clinical-grade imaging agent was manufactured for use in human subjects to identify malignancies in humans in the studies described in detail in the Examples below.

In accordance with a further aspect, the present teachings include methods for the use of the imaging agent to identify malignancies in the bone marrow that include injection of the imaging agent followed by PET imaging. In one embodiment, imaging occurs from about one to about six hours after injection of the imaging agent. In some embodiments, the human subject is a multiple myeloma patient.

In various additional aspects, methods to image malignant bone marrow using the disclosed imaging agents are disclosed herein. The method includes injecting a patient with a clinical dose of the imaging agent and performing PET imaging to spatially identify lesions in the bone marrow based on PET signals produced by radionuclides of the imaging agent that are preferentially bound to VLA4 in the bone marrow at the targeting ligand T of the imaging agent. In one embodiment, the PET imaging occurs from about one hour after injection to about six hours after injection of the imaging agent. In some embodiments, this imaging occurs after injecting the imaging agent radiolabeled with the radionuclide ⁶⁴Cu, whose 12.7 hour half-life makes it an ideal radionuclide for this post-injection imaging window.

In another aspect, PET imaging using the disclosed imaging agent is used to select a treatment for a bone marrow malignancy including, but not limited to, multiple myeloma, by injecting the imaging agent, performing subsequent PET imaging, and making a treatment decision based on the imaging results. In some aspects, a method to select a treatment for a bone marrow malignancy patient is disclosed that includes injecting a patient with a clinical dose of the imaging agent, performing PET imaging on the patient, quantifying VLA4 expression in the bone marrow from the PET images, and identifying patients who will respond to therapies based on a high/low expression stratification of patients. In one aspect, this method is used to select treatment when the malignancy is multiple myeloma. In one exemplary embodiment, PET imaging occurs from about one hour to about six hours after injection of the imaging agent.

In yet another aspect, PET imaging may be used to monitor relapse in bone marrow malignancy patients, including but not limited to multiple myeloma patients, by identifying residual disease post-treatment or the appearance of recurrence over time, including recurrent refractory disease in multiple myeloma. In some aspects, a method to monitor for relapse in a bone marrow malignancy patient is disclosed that includes the PET imaging methods described above (injection of a clinical dose and subsequent imaging) performed serially pre-treatment, post-treatment, and at subsequent time points to monitor the change in VLA4 expression over time, wherein a significant increase in VLA4 expression between the post-treatment values and subsequent values is indicative of relapse. In one aspect, the patient is a multiple myeloma patient. In other aspects, PET imaging occurs from about one hour to about six hours after injection of the imaging agent for each sequential PET imaging session.

In yet another embodiment, a systems biology approach is incorporated to integrate clinical data with the imaging results to better inform treatment options.

The present teachings also include compositions and methods of manufacturing kits containing the imaging agent and other components configured to form a dose of an imaging agent ready for injection in a human subject as part of an imaging procedure.

In various other aspects, kits configured to produce and provide human doses of the disclosed imaging agent are disclosed herein. The kits comprise the imaging agent described above, synthesized with GMP, other safe stabilizing agents including but not limited to ascorbic acid and albumin, and packaging in the form of vials that can be easily accessed by clinicians to deliver doses to human patients in clinical settings. In some aspects, the walls of the vials may be coated or functionalized to facilitate the storage and use of the kits to produce and provide human doses of the disclosed imaging agent.

In additional aspects, methods to manufacture and package the disclosed kits are disclosed herein. These kits may be provided in non-radioactive and radioactive forms. For the non-radioactive kits, the non-radioactive peptide portion (T-L-B or T-B) described above is packaged in a vial wherein the radionuclide can be easily added to the sample for simple radiolabeling of the imaging agent for subsequent use in a human subject in a clinical setting. For the radioactive kit form, the radiolabeled imaging agent (T-L-B--R or T-B--R) is packaged in vials in a composition configured to be directly withdrawn from the vial and injected into human subjects.

The landscape of multiple myeloma changes unreliably in a majority of patients. Tumor heterogeneity, development of resistance to drugs, relapsed or refractory disease, the persistence of minimal residual disease, and variability in response are some of the key hallmarks of multiple myeloma. Molecular imaging can address some of the critical issues in the management of multiple myeloma patients such as the accurate establishment of disease burden spatially, unambiguous staging, and quantitative and qualitative assessment of sites of residual disease. Identification of reliable molecular markers during the course of the disease is sought for effectively managing myeloma patients. Some of the outstanding challenges that remain in the myeloma diagnostic tool kit are—1) tissue biopsies are prone to sampling errors, 2) serum assays can be confounding in the cases of oligoseretory and heavily treated myelomas, 3) X-ray modalities are not sensitive to myeloma induced osteolytic lesions, and 4) FDG/PET has inherent limitations in multiple myeloma. Therefore, sensitive imaging techniques such as PET using molecularly targeted imaging agents are fundamentally poised to meet these needs. In recent years, different groups have assessed the utility of myeloma-specific agents targeted to myeloma proteins such as CD38 and CXCR4. Metabolic tracers such as ¹¹C-acetate have also been explored as alternatives to FDG.

The involvement of bone and bone marrow is integral to myeloma origin, pathogenesis, and the development of multi-drug resistance. Strikingly, over 80% of myeloma patients present with skeletal-related events and an unfortunately large number suffer from pathological fractures. Therefore, focusing on the markers that are involved in the adherence, survival, and progression of myeloma cells to the bone marrow is highly relevant. Multiple myeloma cells interact with the VCAM-1 expressed on the bone marrow stromal cells and soluble fibronectin via VLA4. This interplay contributes to reduced osteoblastogenesis and increased osteoclastogenesis, ultimately causing weakening of the bone and progression of multiple myeloma.

### VLA4 Modulation Agents

As described herein, VLA4 expression has been implicated in various diseases, disorders, and conditions. As such, modulation of VLA4 (e.g., modulation of VLA4) can be used for the treatment of such conditions. A VLA4 modulation agent can modulate VLA4 activation response or induce or inhibit VLA4 receptor expression. VLA4 modulation can comprise modulating the expression of VLA4 on cells, modulating the quantity of cells that express VLA4, or modulating the quality of the VLA4 receptor on cells.

VLA4 modulation agents can be any composition or method that can modulate VLA4 expression on cells (e.g., increasing or decreasing VLA4 receptor expression on cells in malignancies in the bone marrow). For example, a VLA4 modulation agent can be an activator, an inhibitor, an agonist, or an antagonist. As another example, the VLA4 modulation can be the result of gene editing.

A VLA4 modulation agent can be a VLA4 antibody (e.g., a monoclonal antibody to the VLA4 receptor).

A VLA4-modulating agent can be an agent that induces or inhibits progenitor cell differentiation into VLA4-expressing cells (e.g., immune cells in the bone marrow). For example, the agent can be used to enhance or inhibit the expression of VLA on immune cells of various types in the bone marrow.

VLA4 Signal Reduction, Elimination, or Inhibition by Small Molecule Inhibitors, shRNA, siRNA, or ASOs

As described herein, a VLA4 modulation agent can be used for use in cancer therapy. A VLA4 modulation agent can be used to reduce/eliminate or enhance/increase VLA4 expression and signals. For example, a VLA4 modulation agent can be a small molecule inhibitor of VLA4. As another example, a VLA4 modulation agent can be a short hairpin RNA (shRNA). As another example, a VLA4 modulation agent can be a short interfering RNA (siRNA).

As another example, RNA (e.g., long noncoding RNA (lncRNA)) can be targeted with antisense oligonucleotides (ASOs) as a therapeutic. Processes for making ASOs targeted to RNAs are well known; see e.g. Zhou et al. 2016 Methods Mol Biol. 1402:199-213. Except as otherwise noted herein, therefore, the process of the present disclosure can be carried out in accordance with such processes.

### VLA4 Inhibiting Agent

One aspect of the present disclosure provides for targeting of VLA4, its receptor, or its downstream signaling. The present disclosure provides methods of treating or preventing VLA4-based progression in bone marrow malignancies based on the discovery that VLA4 expression has been linked to progression in multiple myeloma.

As described herein, inhibitors of VLA4 (e.g., antibodies, fusion proteins, small molecules) can reduce or prevent progression and recurrence in bone marrow malignancies. A VLA4 inhibiting agent can be any agent that can inhibit VLA4, downregulate VLA4, or knockdown VLA4.

As an example, a VLA4 inhibiting agent can inhibit VLA4 signaling.

For example, the VLA4 inhibiting agent can be an anti-VLA4 antibody. Furthermore, the anti-VLA4 antibody can be a murine antibody, a humanized murine antibody, or a human antibody.

As another example, the VLA4 inhibiting agent can be an anti-VLA4 antibody, wherein the anti-VLA4 antibody prevents binding of VLA4 to its receptor, vascular cell adhesion molecule 1 (VCAM), or prevents activation of VLA4 and downstream signaling.

As another example, the VLA4 inhibiting agent can be a fusion protein. For example, the fusion protein can be a decoy receptor for VLA4. Furthermore, the fusion protein can comprise a mouse or human Fc antibody domain fused to the ectodomain of VCAM and/or VLA4.

As another example, a VLA4 inhibiting agent can be Natalizumab, which has been shown to be a potent and specific inhibitor of VLA4 signaling.

As another example, a VLA4 inhibiting agent can be an inhibitory protein that antagonizes VLA4. For example, the VLA4 inhibiting agent can be a viral protein, which has been shown to antagonize VLA4.

As another example, a VLA4 inhibiting agent can be a short hairpin RNA (shRNA) or a short interfering RNA (siRNA) targeting VLA4 or VCAM.

As another example, a VLA4 inhibiting agent can be a sgRNA targeting VLA4 or VCAM.

Methods for preparing a VLA4 inhibiting agent (e.g., an agent capable of inhibiting VLA4 signaling) can comprise the construction of a protein/Ab scaffold containing the natural VLA4 receptor as a VLA4 neutralizing agent; developing inhibitors of the VLA4 receptor “down-stream”; or developing inhibitors of the VLA4 production “up-stream”.

Inhibiting VLA4 can be performed by genetically modifying VLA4 in a subject or genetically modifying a subject to reduce or prevent expression of the VLA4 gene, such as through the use of CRISPR-Cas9 or analogous technologies, wherein, such modification reduces or prevents VLA4.

### Chemical Agent:

Examples of VLA4 targeting agents are described herein. VLA4 targeting agents can be include a formula that includes R groups that may be optionally substituted with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; a C₂₋₁₀cycloalkyl optionally containing unsaturation or one oxygen or nitrogen atom; straight chain or branched C₁₋₁₀alkyl amine; heterocyclyl; heterocyclic amine; and aryl comprising a phenyl; heteroaryl containing from 1 to 4 N, O, or S atoms; unsubstituted phenyl ring; substituted phenyl ring; unsubstituted heterocyclyl; and substituted heterocyclyl, wherein the unsubstituted phenyl ring or substituted phenyl ring can be optionally substituted with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; straight chain or branched C₁₋₁₀alkyl amine, optionally containing unsaturation; a C₂₋₁₀cycloalkyl optionally containing unsaturation or one oxygen or nitrogen atom; straight chain or branched C₁₋₁₀alkyl amine; heterocyclyl; heterocyclic amine; aryl comprising a phenyl; and heteroaryl containing from 1 to 4 N, O, or S atoms; and the unsubstituted heterocyclyl or substituted heterocyclyl can be optionally substituted with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; straight chain or branched C₁₋₁₀alkyl amine, optionally containing unsaturation; a C₂₋₁₀cycloalkyl optionally containing unsaturation or one oxygen or nitrogen atom; heterocyclyl; straight chain or branched C₁₋₁₀alkyl amine; heterocyclic amine; and aryl comprising a phenyl; and heteroaryl containing from 1 to 4 N, O, or S atoms. Any of the above can be further optionally substituted.

The term “imine” or “imino”, as used herein, unless otherwise indicated, can include a functional group or chemical compound containing a carbon-nitrogen double bond. The expression “imino compound”, as used herein, unless otherwise indicated, refers to a compound that includes an “imine” or an “imino” group as defined herein. The “imine” or “imino” group can be optionally substituted.

The term “hydroxyl”, as used herein, unless otherwise indicated, can include-OH. The “hydroxyl” can be optionally substituted.

The terms “halogen” and “halo”, as used herein, unless otherwise indicated, include a chlorine, chloro, Cl; fluorine, fluoro, F; bromine, bromo, Br; or iodine, iodo, or I.

The term “acetamide”, as used herein, is an organic compound with the formula CH₃CONH₂. The “acetamide” can be optionally substituted.

The term “aryl”, as used herein, unless otherwise indicated, include a carbocyclic aromatic group. Examples of aryl groups include, but are not limited to, phenyl, benzyl, naphthyl, or anthracenyl. The “aryl” can be optionally substituted.

The terms “amine” and “amino”, as used herein, unless otherwise indicated, include a functional group that contains a nitrogen atom with a lone pair of electrons and wherein one or more hydrogen atoms have been replaced by a substituent such as, but not limited to, an alkyl group or an aryl group. The “amine” or “amino” group can be optionally substituted.

The term “alkyl”, as used herein, unless otherwise indicated, can include saturated monovalent hydrocarbon radicals having straight or branched moieties, such as but not limited to, methyl, ethyl, propyl, butyl, pentyl, hexyl, octyl groups, etc. Representative straight-chain lower alkyl groups include, but are not limited to, -methyl, -ethyl, -n-propyl, -n-butyl, -n-pentyl, -n-hexyl, -n-heptyl and -n-octyl; while branched lower alkyl groups include, but are not limited to, -isopropyl, -sec-butyl, -isobutyl, -tert-butyl, -isopentyl, 2-methylbutyl, 2-methylpentyl, 3-methylpentyl, 2,2-dimethylbutyl, 2,3-dimethylbutyl, 2,2-dimethylpentyl, 2,3-dimethylpentyl, 3,3-dimethylpentyl, 2,3,4-trimethylpentyl, 3-methylhexyl, 2,2-dimethylhexyl, 2,4-dimethylhexyl, 2,5-dimethylhexyl, 3,5-dimethylhexyl, 2,4-dimethylpentyl, 2-methylheptyl, 3-methylheptyl, unsaturated C₁₋₁₀ alkyls include, but are not limited to, -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, 1-hexyl, 2-hexyl, 3-hexyl, -acetylenyl, -propynyl, -1-butynyl, -2-butynyl, -1-pentynyl, -2-pentynyl, or 3-methyl-1 butynyl. An alkyl can be saturated, partially saturated, or unsaturated. The “alkyl” can be optionally substituted.

The term “carboxyl”, as used herein, unless otherwise indicated, can include a functional group consisting of a carbon atom double-bonded to an oxygen atom and single-bonded to a hydroxyl group (—COOH). The “carboxyl” can be optionally substituted.

The term “alkenyl”, as used herein, unless otherwise indicated, can include alkyl moieties having at least one carbon-carbon double bond wherein alkyl is as defined above and including E and Z isomers of said alkenyl moiety. An alkenyl can be partially saturated or unsaturated. The “alkenyl” can be optionally substituted.

The term “alkynyl”, as used herein, unless otherwise indicated, can include alkyl moieties having at least one carbon-carbon triple bond wherein alkyl is as defined above. An alkynyl can be partially saturated or unsaturated. The “alkynyl” can be optionally substituted.

The term “acyl”, as used herein, unless otherwise indicated, can include a functional group derived from an aliphatic carboxylic acid, by removal of the hydroxyl (—OH) group. The “acyl” can be optionally substituted.

The term “alkoxyl”, as used herein, unless otherwise indicated, can include O-alkyl groups wherein alkyl is as defined above and O represents oxygen. Representative alkoxyl groups include, but are not limited to, —O-methyl, —O-ethyl, —O-n-propyl, —O-n-butyl, —O-n-pentyl, —O-n-hexyl, —O-n-heptyl, —O-n-octyl, —O-isopropyl, —O-sec-butyl, —O-isobutyl, —O-tert-butyl, —O-isopentyl, —O-2-methylbutyl, —O-2-methylpentyl, —O-3-methylpentyl, —O-2,2-dimethylbutyl, —O-2,3-dimethylbutyl, —O-2,2-dimethylpentyl, —O-2,3-dimethylpentyl, —O-3,3-dimethylpentyl, —O-2,3,4-trimethylpentyl, —O-3-methylhexyl, —O-2,2-dimethylhexyl, —O-2,4-dimethylhexyl, —O-2,5-dimethylhexyl, —O-3,5-dimethylhexyl, —O-2,4dimethylpentyl, —O-2-methylheptyl, —O-3-methylheptyl, —O-vinyl, —O-allyl, —O-1-butenyl, —O-2-butenyl, —O-isobutylenyl, —O-1-pentenyl, —O-2-pentenyl, —O-3-methyl-1-butenyl, —O-2-methyl-2-butenyl, —O-2,3-dimethyl-2-butenyl, —O-1-hexyl, —O-2-hexyl, —O-3-hexyl, —O-acetylenyl, —O-propynyl, —O-1-butynyl, —O-2-butynyl, —O-1-pentynyl, —O-2-pentynyl and —O-3-methyl-1-butynyl, —O-cyclopropyl, —O-cyclobutyl, —O-cyclopentyl, —O-cyclohexyl, —O-cycloheptyl, —O-cyclooctyl, —O-cyclononyl and —O-cyclodecyl, —O-CH₂-cyclopropyl, —O-CH₂-cyclobutyl, —O-CH₂-cyclopentyl, —O-CH₂-cyclohexyl, —O-CH₂-cycloheptyl, —O-CH₂-cyclooctyl, —O-CH₂-cyclononyl, —O-CH₂-cyclodecyl, —O—(CH₂)₂-cyclopropyl, —O—(CH₂)₂-cyclobutyl, —O-(CH₂)₂-cyclopentyl, —O—(CH₂)₂-cyclohexyl, —O—(CH₂)₂-cycloheptyl, —O—(CH₂)₂-cyclooctyl, —O—(CH₂)₂-cyclononyl, or —O—(CH₂)₂-cyclodecyl. An alkoxyl can be saturated, partially saturated, or unsaturated. The “alkoxyl” can be optionally substituted.

The term “cycloalkyl”, as used herein, unless otherwise indicated, can include an aromatic, non-aromatic, saturated, partially saturated, or unsaturated, monocyclic or fused, spiro or unfused bicyclic or tricyclic hydrocarbon referred to herein containing a total of from 1 to 10 carbon atoms (e.g., 1 or 2 carbon atoms if there are other heteroatoms in the ring), preferably 3 to 8 ring carbon atoms. Examples of cycloalkyls include, but are not limited to, C₃₋₁₀ cycloalkyl groups include, but are not limited to, -cyclopropyl, -cyclobutyl, -cyclopentyl, -cyclopentadienyl, -cyclohexyl, -cyclohexenyl, -1,3-cyclohexadienyl, -1,4-cyclohexadienyl, -cycloheptyl, -1,3-cycloheptadienyl, -1,3,5-cycloheptatrienyl, -cyclooctyl, and -cyclooctadienyl. The term “cycloalkyl” also can include-lower alkyl-cycloalkyl, wherein lower alkyl and cycloalkyl are as defined herein.

Examples of -lower alkyl-cycloalkyl groups include, but are not limited to, —CH₂-cyclopropyl, —CH₂-cyclobutyl, —CH₂-cyclopentyl, —CH₂-cyclopentadienyl, —CH₂-cyclohexyl, —CH₂-cycloheptyl, or —CH₂-cyclooctyl. The “cycloalkyl” can be optionally substituted. A “cycloheteroalkyl”, as used herein, unless otherwise indicated, can include any of the above with a carbon substituted with a heteroatom (e.g., O, S, N).

The term “heterocyclic” or “heteroaryl”, as used herein, unless otherwise indicated, can include an aromatic or non-aromatic cycloalkyl in which one to four of the ring carbon atoms are independently replaced with a heteroatom from the group consisting of O, S and N. Representative examples of a heterocycle include, but are not limited to, benzofuranyl, benzothiophene, indolyl, benzopyrazolyl, coumarinyl, isoquinolinyl, pyrrolyl, pyrrolidinyl, thiophenyl, furanyl, thiazolyl, imidazolyl, pyrazolyl, triazolyl, quinolinyl, pyrimidinyl, pyridinyl, pyridonyl, pyrazinyl, pyridazinyl, isothiazolyl, isoxazolyl, (1,4)-dioxane, (1,3)-dioxolane, 4,5-dihydro-1H-imidazolyl, or tetrazolyl. Heterocycles can be substituted or unsubstituted. Heterocycles can also be bonded at any ring atom (i.e., at any carbon atom or heteroatom of the heterocyclic ring). A heterocyclic can be saturated, partially saturated, or unsaturated. The “heterocyclic” can be optionally substituted.

The term “indole”, as used herein, is an aromatic heterocyclic organic compound with the formula C₈H₇N. It has a bicyclic structure, consisting of a six-membered benzene ring fused to a five-membered nitrogen-containing pyrrole ring. The “indole” can be optionally substituted.

The term “cyano”, as used herein, unless otherwise indicated, can include a-CN group. The “cyano” can be optionally substituted.

The term “alcohol”, as used herein, unless otherwise indicated, can include a compound in which the hydroxyl functional group (—OH) is bound to a carbon atom. In particular, this carbon center should be saturated, having single bonds to three other atoms. The “alcohol” can be optionally substituted.

The term “solvate” is intended to mean a solvate form of a specified compound that retains the effectiveness of such a compound. Examples of solvates include compounds of the invention in combination with, for example: water, isopropanol, ethanol, methanol, dimethylsulfoxide (DMSO), ethyl acetate, acetic acid, or ethanolamine.

The term “mmol”, as used herein, is intended to mean millimole. The term “equiv”, as used herein, is intended to mean equivalent. The term “mL”, as used herein, is intended to mean milliliter. The term “g”, as used herein, is intended to mean gram. The term “kg”, as used herein, is intended to mean kilogram. The term “μg”, as used herein, is intended to mean micrograms. The term “h”, as used herein, is intended to mean hour. The term “min”, as used herein, is intended to mean minute. The term “M”, as used herein, is intended to mean molar. The term “μL”, as used herein, is intended to mean microliter. The term “μM”, as used herein, is intended to mean micromolar. The term “nM”, as used herein, is intended to mean nanomolar. The term “N”, as used herein, is intended to mean normal. The term “amu”, as used herein, is intended to mean atomic mass unit. The term “° C.”, as used herein, is intended to mean degree Celsius. The term “wt/wt”, as used herein, is intended to mean weight/weight. The term “v/v”, as used herein, is intended to mean volume/volume. The term “MS”, as used herein, is intended to mean mass spectroscopy. The term “HPLC”, as used herein, is intended to mean high-performance liquid chromatograph. The term “RT”, as used herein, is intended to mean room temperature. The term “e.g.”, as used herein, is intended to mean example. The term “N/A”, as used herein, is intended to mean not tested.

As used herein, the expression “pharmaceutically acceptable salt” refers to pharmaceutically acceptable organic or inorganic salts of a compound of the invention. Preferred salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, or pamoate (i.e., 1,1′-methylene-bis-(2-hydroxy-3-naphthoate)) salts. A pharmaceutically acceptable salt may involve the inclusion of another molecule such as an acetate ion, a succinate ion, or other counterions. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counterions. Hence, a pharmaceutically acceptable salt can have one or more charged atoms and/or one or more counterion. As used herein, the expression “pharmaceutically acceptable solvate” refers to an association of one or more solvent molecules and a compound of the invention. Examples of solvents that form pharmaceutically acceptable solvates include, but are not limited to, water, isopropanol, ethanol, methanol, DMSO, ethyl acetate, acetic acid, and ethanolamine. As used herein, the expression “pharmaceutically acceptable hydrate” refers to a compound of the invention, or a salt thereof, that further can include a stoichiometric or non-stoichiometric amount of water bound by non-covalent intermolecular forces.

### Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce the dosage frequency. Controlled-release preparations can also be used to affect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for the treatment of the disease, disorder, or condition.

### Therapeutic Methods

Also provided is a process of treating, preventing, or reversing bone marrow malignancies, including but not limited to bone marrow-based cancers like multiple myeloma and leukemia and metastases in the bone marrow from other cancers like breast, prostate, and lung cancers, in a subject in need of administration of a therapeutically effective amount of a bone marrow malignancy therapy, so as to inhibit progression, decrease recurrence, and/or extend survival.

Methods described herein are generally performed on a subject in need thereof. A subject in need of the therapeutic methods described herein can be a subject having, diagnosed with, suspected of having, or at risk for developing a bone marrow malignancy. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of therapeutic drug that treats bone marrow malignancies is, for example, an amount that would cause the desired therapeutic effect in a subject while minimizing undesired side effects. In various embodiments, an effective amount of a drug that treats bone marrow malignancies, which include but are not limited to VLA4 inhibitors, described herein can substantially inhibit the appearance or recurrence of bone marrow malignancies, slow progression, or limit the development of drug-resistant recurrence.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of drug that treats bone marrow malignancies can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to inhibit VLA4 expression.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

The toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from the compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treatment can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of a drug that treats bone malignancies can occur as a single event or over a time course of treatment. For example, the drug can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for cancers of various types, including but not limited to those bone marrow-based cancers and metastatic diseases described above.

A drug to treat bone marrow malignancies can be administered simultaneously or sequentially with another agent, such as an antibiotic, an anti-inflammatory, or another agent. For example, a drug can be administered simultaneously with another agent, such as an antibiotic or an anti-inflammatory. Simultaneous administration can occur through the administration of separate compositions, each containing one or more of a cancer drug, an antibiotic, an anti-inflammatory, or another agent. Simultaneous administration can occur through the administration of one composition containing two or more of a cancer drug, an antibiotic, an anti-inflammatory, or another agent. A cancer drug can be administered sequentially with an antibiotic, an anti-inflammatory, or another agent. For example, a cancer drug can be administered before or after the administration of an antibiotic, an anti-inflammatory, or another agent.

### Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well-known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10:0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve the taste of the product; or improve the shelf life of the product.

### Screening

Also provided are methods for screening.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 mw, or less than about 1000 mw, or less than about 800 mw) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example: ChemBridge, Princeton BioMolecular, Ambinter SARL, Enamine, ASDI, Life Chemicals, etc.).

Candidate molecules for screening according to the methods described herein include both lead-like compounds and drug-like compounds. A lead-like compound is generally understood to have a relatively smaller scaffold-like structure (e.g., a molecular weight of about 150 to about 350 kD) with relatively fewer features (e.g., less than about 3 hydrogen donors and/or less than about 6 hydrogen acceptors; hydrophobicity character×log P of about-2 to about 4) (see e.g., Angewante (1999) Chemie Int. ed. Engl. 24, 3943-3948). In contrast, a drug-like compound is generally understood to have a relatively larger scaffold (e.g., a molecular weight of about 150 to about 500 kD) with relatively more numerous features (e.g., less than about 10 hydrogen acceptors and/or less than about 8 rotatable bonds; hydrophobicity character×log P of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number 5 among them). While these rules generally relate to oral absorption and are used to predict bioavailability of compounds during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

### Kits

Also provided are kits. Such kits can include an agent or composition described herein and, in certain embodiments, instructions for administration. Such kits can facilitate the performance of the methods described herein. When supplied as a kit, the different components of the composition can be packaged in separate containers and admixed immediately before use. Components include, but are not limited to a VLA4 targeting agent, albumin, ascorbic acid, and other stabilizing agents. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing the activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium or video. Detailed instructions may not be physically associated with the kit; instead, a user may be directed to an Internet website specified by the manufacturer or distributor of the kit.

A control sample or a reference sample as described herein can be a sample from a healthy subject. A reference value can be used in place of a control or reference sample, which was previously obtained from a healthy subject or a group of healthy subjects. A control sample or a reference sample can also be a sample with a known amount of a detectable compound or a spiked sample.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10:0879697717; Ausubel et al. (2002) Short Protocols in Molecular Biology, 5th ed., Current Protocols, ISBN-10:0471250929; Sambrook and Russel (2001) Molecular Cloning: A Laboratory Manual, 3d ed., Cold Spring Harbor Laboratory Press, ISBN-10:0879695773; Elhai, J. and Wolk, C. P. 1988. Methods in Enzymology 167, 747-754; Studier (2005) Protein Expr Purif. 41 (1), 207-234; Gellissen, ed. (2005) Production of Recombinant Proteins: Novel Microbial and Eukaryotic Expression Systems, Wiley-VCH, ISBN-10:3527310363; Baneyx (2004) Protein Expression Technologies, Taylor & Francis, ISBN-10:0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1

To demonstrate the synthesis of the GMP-grade imaging agents disclosed herein and the use of the disclosed imaging agents in vitro, in vivo, and in the first in human studies, the following experiments were conducted.

### Materials and Methods

LLP2A-CB-TE1A1P (LLP2A) peptide was purchased from Auspep Pyt (Tullamarine Victoria, Australia). All other chemicals were purchased from Sigma Aldrich unless otherwise noted. Copper-64 (t_{1/2}—12.7 h) was purchased from the Washington University School of Medicine. It was produced on a CS-15 biomedical cyclotron at Washington University School of Medicine.

### Synthesis of ⁶⁴Cu-LLP2A for Human PET Imaging.

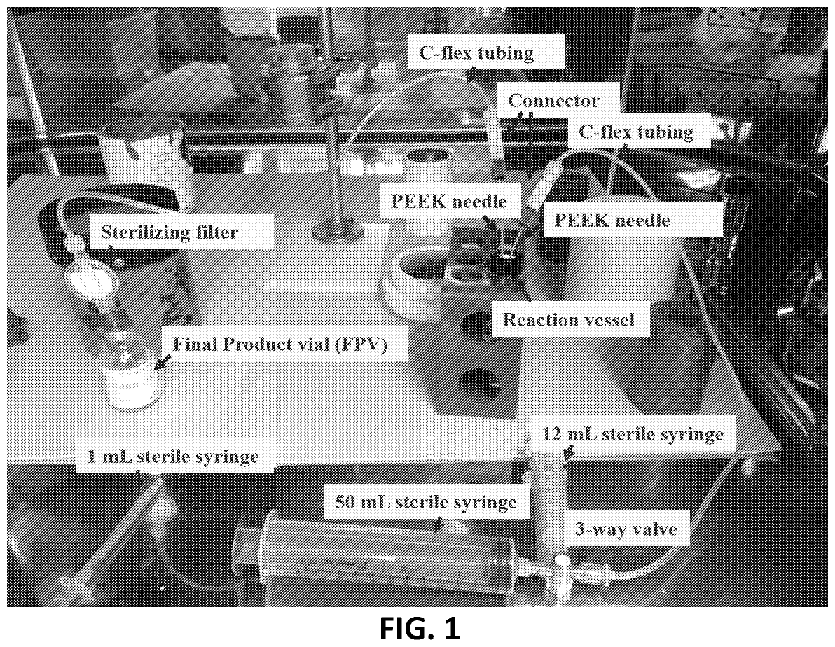

⁶⁴Cu-LLP2A was synthesized according to the good manufacturing practice-compliant procedure in a chemistry hot cell. LLP2A-CB-TE1A1P (15 μg in 65 μL; 0.230 μg/μL) was transferred in a reaction vial with 15 mCi of copper 64 (⁶⁴Cu) and 0.5 M ammonium acetate buffer (pH 6.5) as the radiolabeling buffer. The reaction vial was incubated at 70° C. for 30 min. After the incubation, the reaction vial was removed from the heater block and allowed to cool for 5 min in a lead shield. 0.2 μg/μL gentisic acid in 0.9% sodium chloride solution was added to the reaction vial once the radiolabeling was complete. The set-up for ⁶⁴Cu labeling of LLP2A in a hot cell consisted of the reaction vessel, final product vial (FPV), sterilizing filters, PEEK needles, and C-flex tubing. Two small holes were made in the septum of the reaction vessel with an 18 G needle. The PEEK needle and C-flex tubing were attached to the connector using nylon male and female tube fittings. The PEEK needle was inserted into the reaction vessel through one of the holes created by an 18G needle, and the other end of the C-flex tubing was connected to the sterilizing filter attached to the FPV. 50 mL sterile syringe and a different C-flex tubing were attached to different ends of the 3-way valve. Another PEEK needle was attached to a connector on one end and a 1 mL sterile syringe on the other end. This PEEK needle was inserted into the reaction vessel through the second hole created by the 18G needle. The 1 mL syringe was replaced by the C-flex tubing attached to the 3-way valve after adding ⁶⁴Cu into the reaction vessel. ⁶⁴Cu-LLP2A was transferred to the FPV through the sterilizing filter. An image of the set-up is shown in FIG. 1.

### Pre-Release and Post-Release Quality Control (QC) Specifications.

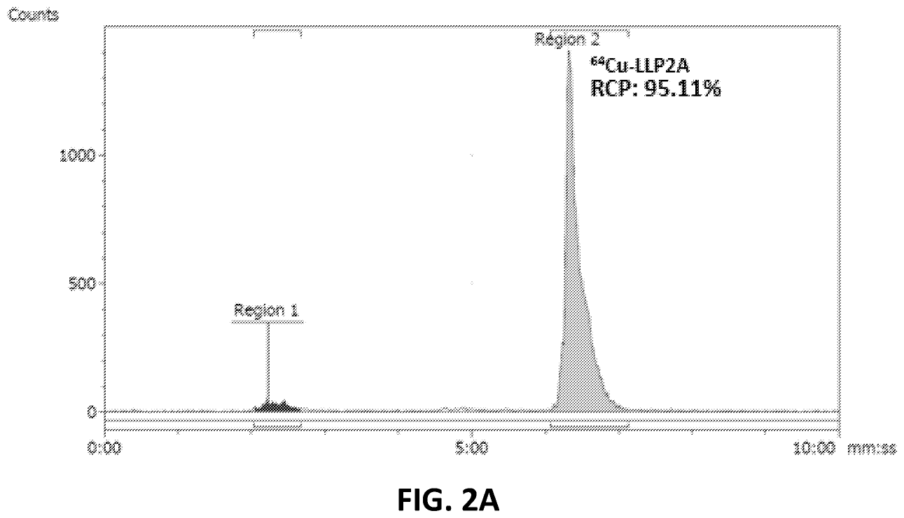

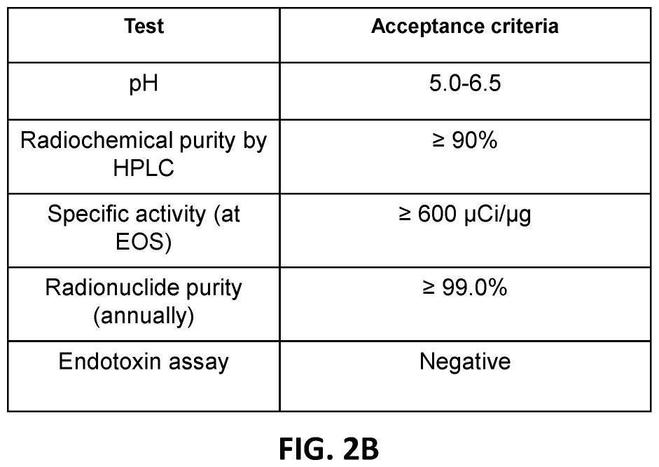

⁶⁴Cu-LLP2A was released for clinical use after it met all pre-release QC release specifications (FIG. 2). Pre-release testing involved radionuclidic identity, filter membrane integrity, visual appearance, pH, radiochemical purity, and bacterial endotoxin tests. The radiochemical purity was evaluated by radio-high-performance liquid chromatography (HPLC). Sterility testing and cell-binding assays were performed after radiotracer release for patient injections.

### Preclinical Studies

### Cell Binding Assays.

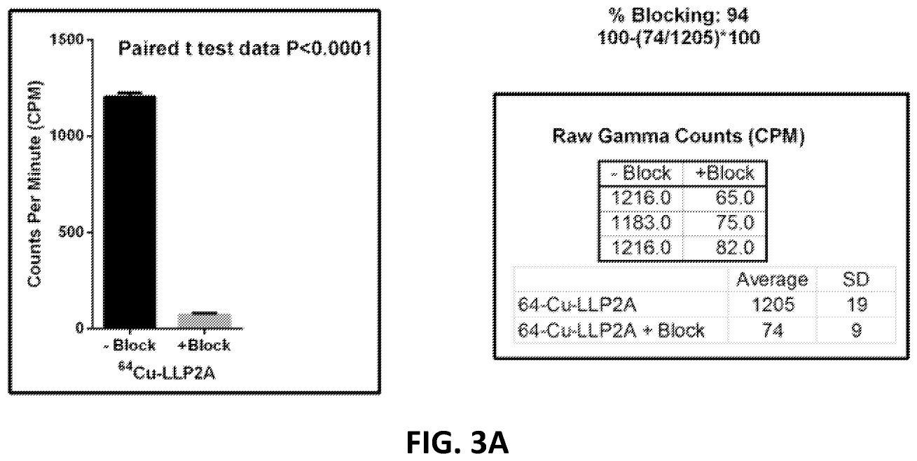

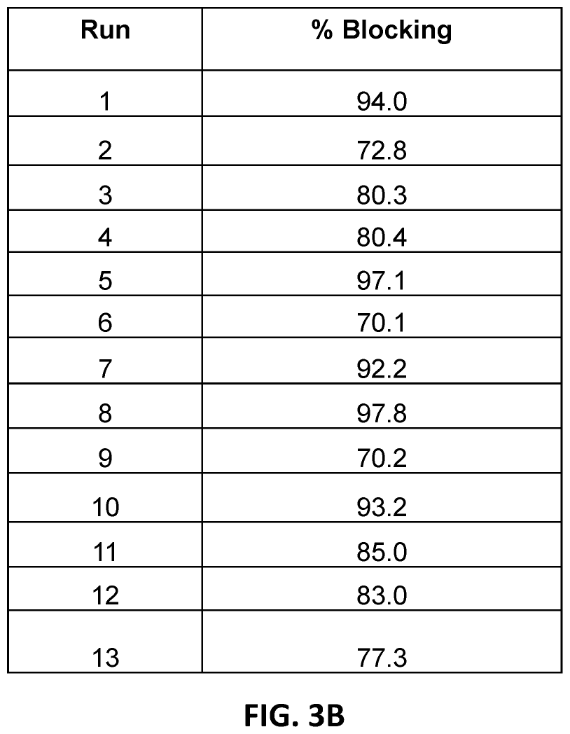

Cell uptake assays were performed to calculate the percentage specific uptake of ⁶⁴Cu-LLP2A in a VLA4 positive myeloma cell line, 5TGM1. Briefly, 5TGM1 murine myeloma cells were grown in IMDM media and allowed to reach confluency. The cells were then harvested, washed, and re-suspended in 1× phosphate-buffered saline (PBS) in acid-washed Eppendorf tubes. A solution of ⁶⁴Cu-LLP2A (0.1 nM) was added to all the tubes. A 200-fold excess of non-radiolabeled LLP2A precursor was added to one set of Eppendorf tubes for blocking. The tubes were incubated at 37° C. for 1 h. After the incubation, samples were centrifuged at 1500 rpm for 3 min, and the supernatant was removed. Cell washing was repeated twice with 1×PBS and the radioactivity in the remaining pellets was measured using a gamma counter (FIG. 3).

### Toxicity Studies in Mice.

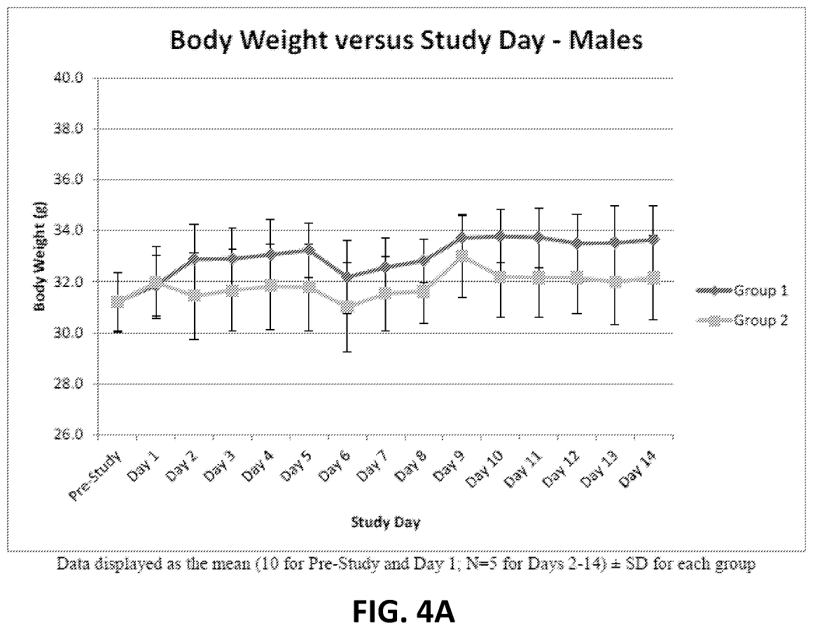

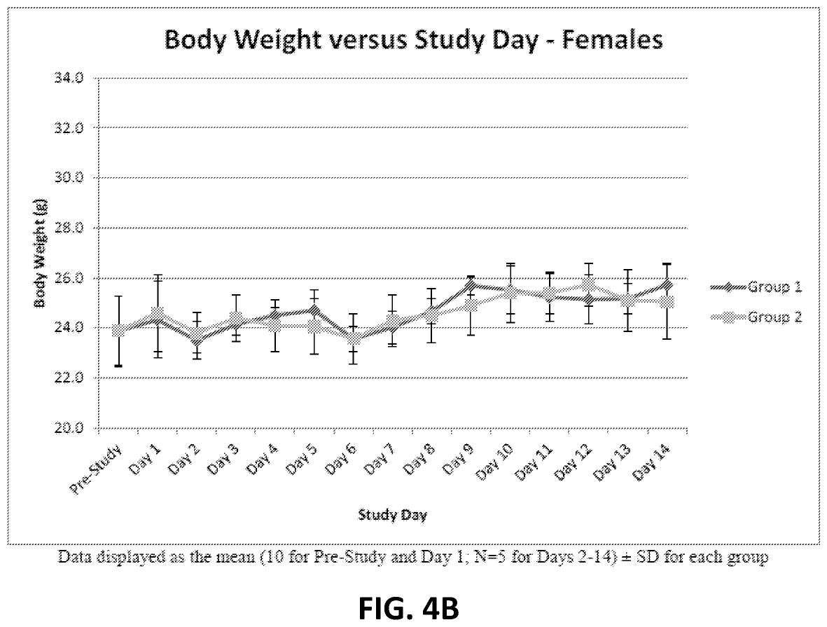

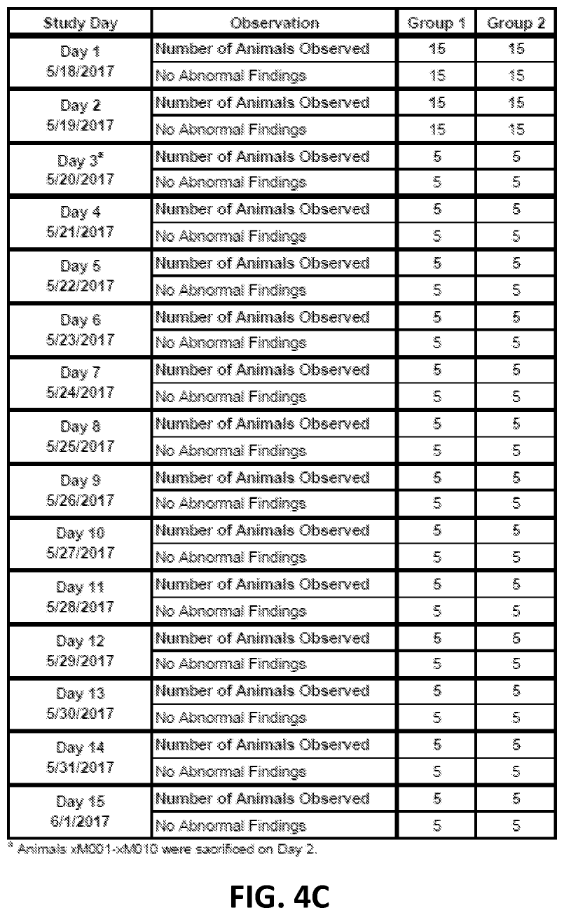

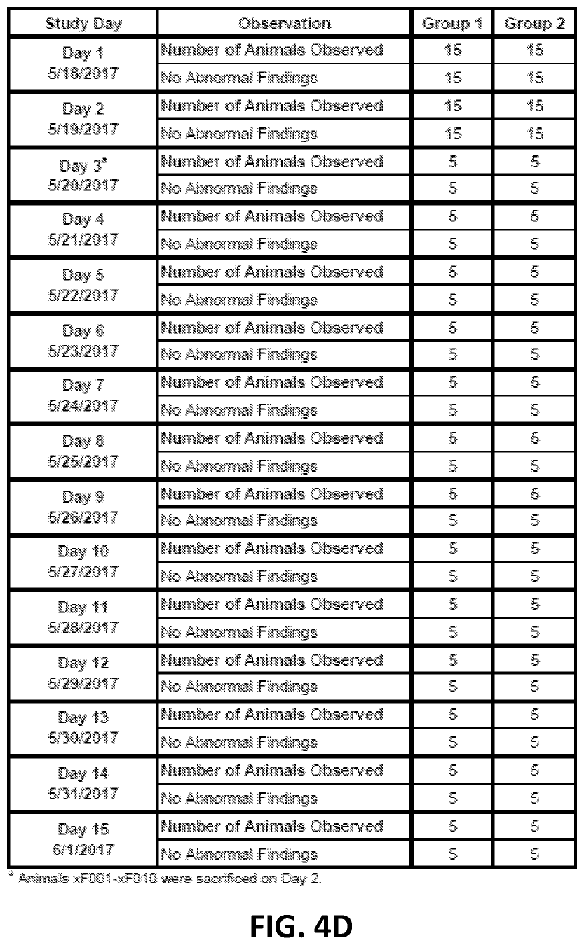

The toxicity of [^{Nat}Cu]-labeled LLP2A was performed in collaboration with Seventh Wave, Maryland Heights (MO). [^{Nat}Cu]Cu-LLP2A was administered once on day 1 in CD-1 IGS mice (n=15 male and n=15 female, respectively). 24 h later (day 2), a group of these mice (n=10/sex/group) was sacrificed and necropsied. Another set of mice (n=5/sex/group) was observed until day 15 and then euthanized. Mice for each time point were divided into two groups—group 1 received vehicle (8% ammonium acetate in water for injection), and group 2 received 0.0103 mg/mouse [^{Nat}Cu]Cu-LLP2A via tail vein at a dose volume of 0.125 mL/mouse. Mice were observed daily and their body weights were daily recorded as well. Blood samples from these mice were collected on day 2 and day 15, respectively, before euthanizing them for necropsy. The blood samples were used to evaluate hematology and clinical chemistry endpoints. Different organs were collected, weighed, and fixed for microscopic evaluation (FIG. 4).

### Clinical Studies

### Patient Population

We studied six healthy volunteers and three subjects with confirmed multiple myeloma diagnoses. This study (NCT03804424) was approved by the Institutional Review Board and the Radioactive Drug Research Committee at Washington University School of Medicine and was conducted under an investigational new drug application (IND #136782) submitted to the U.S. Food and Drug Administration. All patients gave written informed consent before participation. The inclusion criteria for patients with multiple myeloma included adult patients 18 years of age or older with clinically or pathologically defined multiple myeloma in accordance with the International Myeloma Working Group (IMWG) or as stated in the office note/clinical assessment from the treating physician. All types of active myeloma were eligible including both newly diagnosed and previously treated.

Healthy volunteer subject eligibility criteria included adults 18 years of age or older with no known hematologic disorder such as anemia, leukemia, etc. who was considered healthy based on the assessment by the study PI. Patients were housed in a Facility and total urine volume and activity were collected.

### PET Imaging Procedures.

PET imaging was performed with a Siemens mMR, Siemens Biograph 40HD PET/CT, or Siemens Biograph Vision scanners. Subjects entered into the study were asked to undergo ⁶⁴Cu-LLP2A-PET imaging at up to three separate time points for purposes of calculating human dosimetry. Three subjects underwent a 60-min dynamic study immediately after administration of ⁶⁴Cu-LLP2A over the known site of the tumor (typically the biopsied lesion) or over the lower lumbar spine and pelvis (in healthy volunteers or 2 subjects without lesions). Dynamic imaging was followed by two body imaging time points and was used in calculating human dosimetry tables. The remaining 6 patients underwent body imaging at 3 time points between 0 and 26 hrs post injections and did not undergo dynamic imaging. ⁶⁴Cu-LLP2A-PET/MR or PET/CT images were evaluated to determine the imaging time after administration of ⁶⁴Cu-LLP2A that yields the best quality images and the best tumor-to-non-tumor ratio for visual and quantitative analysis of the images. ⁶⁴Cu-LLP2A-PET images also were correlated with all available imaging studies to assess lesion uptake of ⁶⁴Cu-LLP2A in known lesions seen in the radiological studies. In healthy volunteers, the images were evaluated to understand the normal biodistribution and the behavior of this tracer in normal organs.

Patients were asked to void their bladder when total urine volume and urine activity concentration were collected before each imaging session. Urine was also collected and counted for radioactivity at several time points for 24 hrs following injection.

Eight subjects entered on study underwent ⁶⁴Cu-LLP2A-PET/MR (Siemens Biograph mMR) and one subject underwent PET/CT imaging (Siemens Biograph Vision) at 3 time points. All Subjects were injected with up to 11.7 mCi (6.96-11.7) of ⁶⁴Cu-LLP2A and underwent whole-body imaging 0-26 hrs following administration of ⁶⁴Cu-LLP2A to study tracer biodistribution and calculate human dosimetry.

PET/MR in all subjects consisted of a 2-point DIXON MRI for attenuation correction and body emission scans (2-5 min per bed position) were performed. For all subjects with multiple myeloma who underwent simultaneous PET/MRI, additional sequences of T1-weighted turbo-spin echo (TSE), T2-weighted fat suppression post-contrast images, Diffusion-weighted imaging (DWI)/apparent diffusion coefficient (ADC) Dynamic imaging contrast-enhanced (DCE) imaging were performed.

PET/CT consisted of a spiral CT scan for attenuation correction (120 kVp, 50 effective mAs at 4-mm slice thickness) from the top of the skull through the upper thighs with the subject in the supine position.

Immediately after the attenuation CT or MR, emission images beginning at the skull and proceeding down through the lower thighs were obtained (1-10 min per bed position depending on the time post-injection) over 6-7 bed positions with a total imaging duration of no more than 1 h. Images were reconstructed with 3D-OSEM with 3 iterations, 21 subsets, and a post-reconstruction Gaussian filter of 4 mm.

For safety evaluation, all patients underwent vital sign measurement (blood pressure, heart and respiratory rate, and temperature), clinical laboratory testing (standard hematologic and comprehensive metabolic panels that included hemoglobin, white blood cells, neutrophils, lymphocytes, platelets, creatinine, blood urea nitrogen, calcium, sodium, potassium, carbon dioxide, alanine transaminase, aspartate aminotransferase, alkaline phosphatase, total bilirubin, and albumin), urinalysis, and electrocardiogramafore ⁶⁴Cu-LLP2A administration, as well as during and after completion of imaging. All subjects also were monitored for adverse reactions (e.g., dyspnea, chest tightness, fever, rigors, etc.) during the administration of ⁶⁴Cu-LLP2A.

### Image Analysis.

PET images of the normal subjects were evaluated qualitatively to assess the biodistribution of ⁶⁴Cu-LLP2A. The images of the patients with myeloma were evaluated qualitatively in comparison with the healthy volunteers with the following grading scale: no uptake (tumor≤background), minimal uptake (tumor=background), moderate uptake (tumor>background), and intense uptake (tumor>>background). The images were evaluated semi-quantitatively by measurement of the tumor maximum standardized uptake value (SUVₘₐₓ). A region of interest (ROI) was drawn around the entire lesion, with knowledge of the tumor location. In patients with no focal lesions and positive bone marrow biopsy of the iliac bone for multiple myeloma, SUVₘₐₓ, SUVₘₑₐₙ, and the iliac bone (or tumor)-to-spleen ratio (and to the liver) were determined.

For radiation dosimetry estimation, VOIs were traced on the organs on the PET images with visible uptake. The liver, spleen, and kidneys average organ activity concentration were measured by drawing VOI that encompass most of the organs as visible on the PET images at each imaging time point. The blood pool activity was measured from a VOI traced with the left ventricle of the heart. Red marrow activity concentration was measured from the VOIs tracer on the marrow uptake seen on the lumbar vertebrae 2-3-4. The total organ activity was then scaled by the standard male or female organ masses as defined in ICRP. Total urinary bladder content was measured from a VOI encompassing the whole bladder as seen on the PET images.

### Organ Time-Integrated Activity and Radiation Dose.

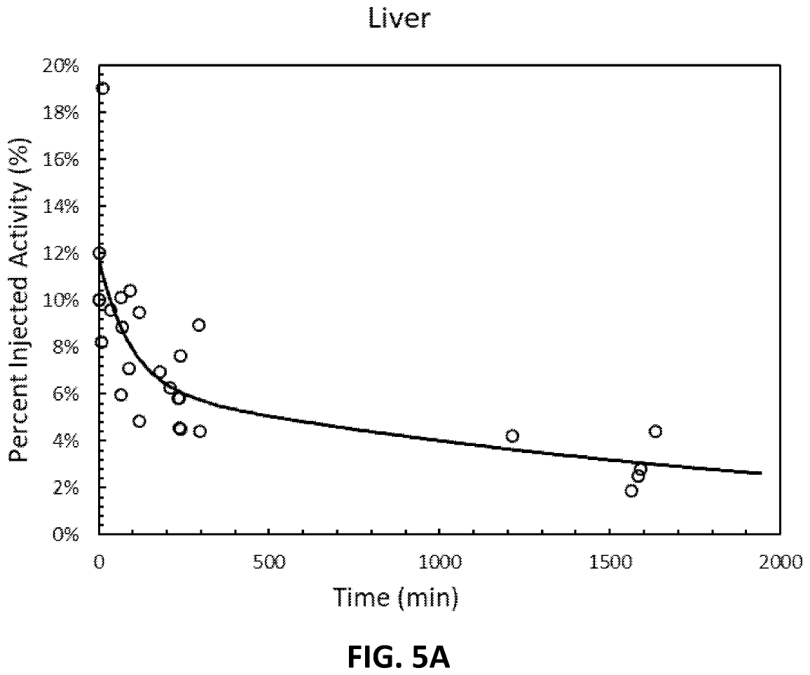

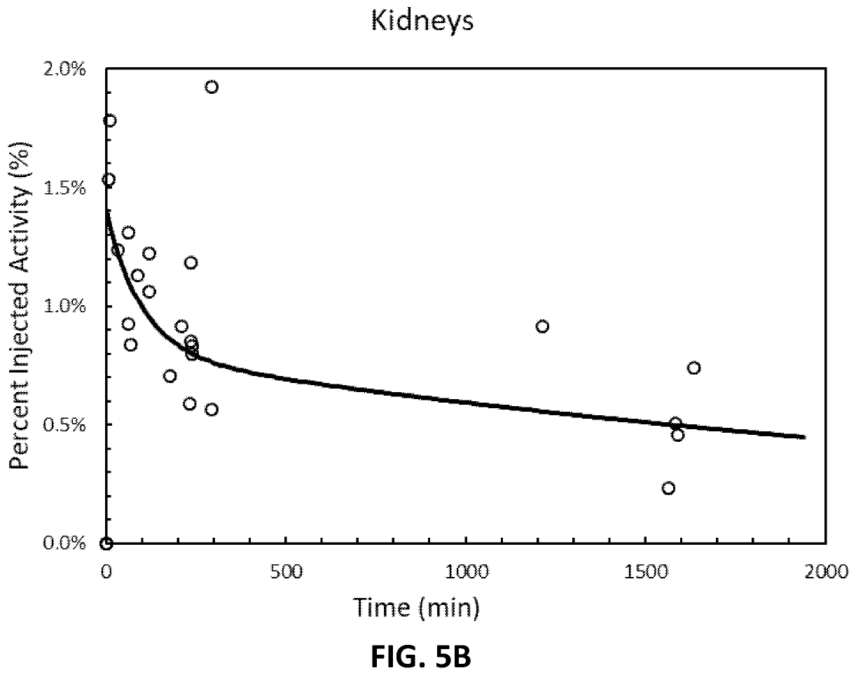

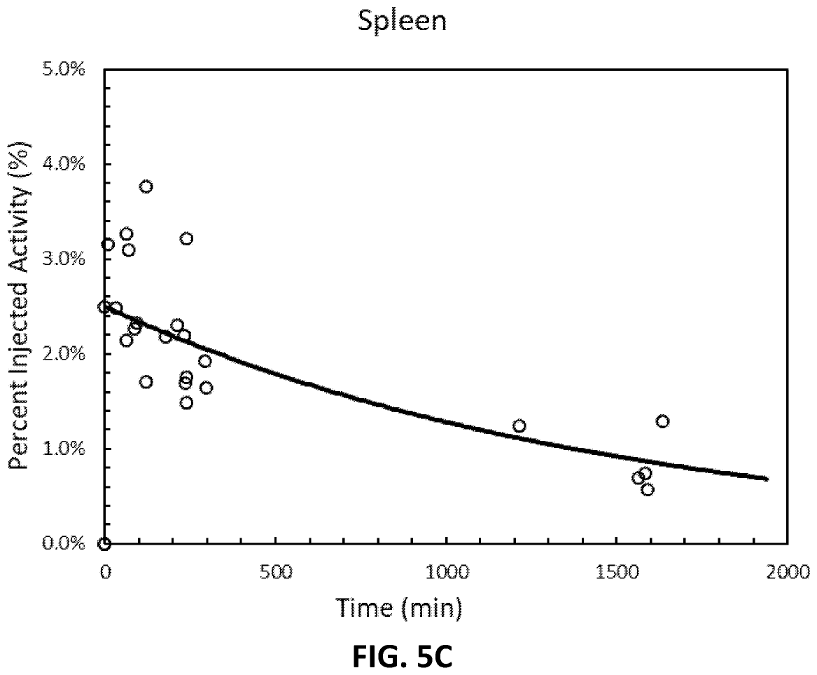

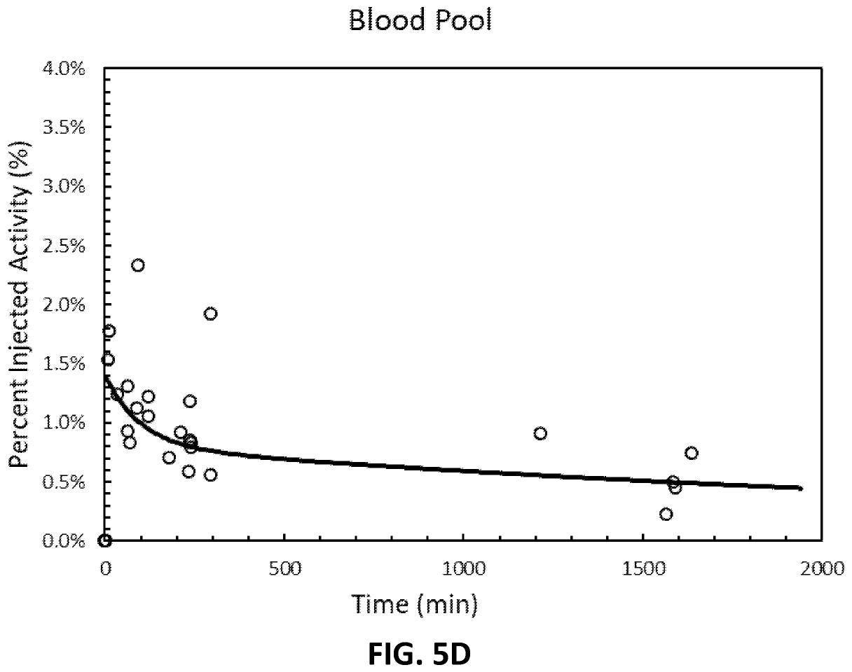

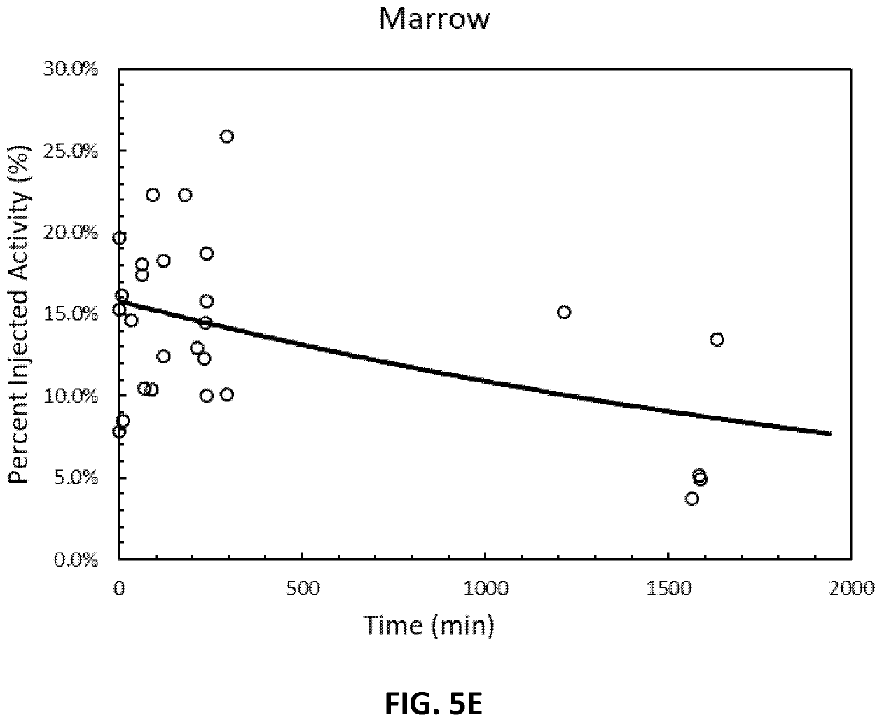

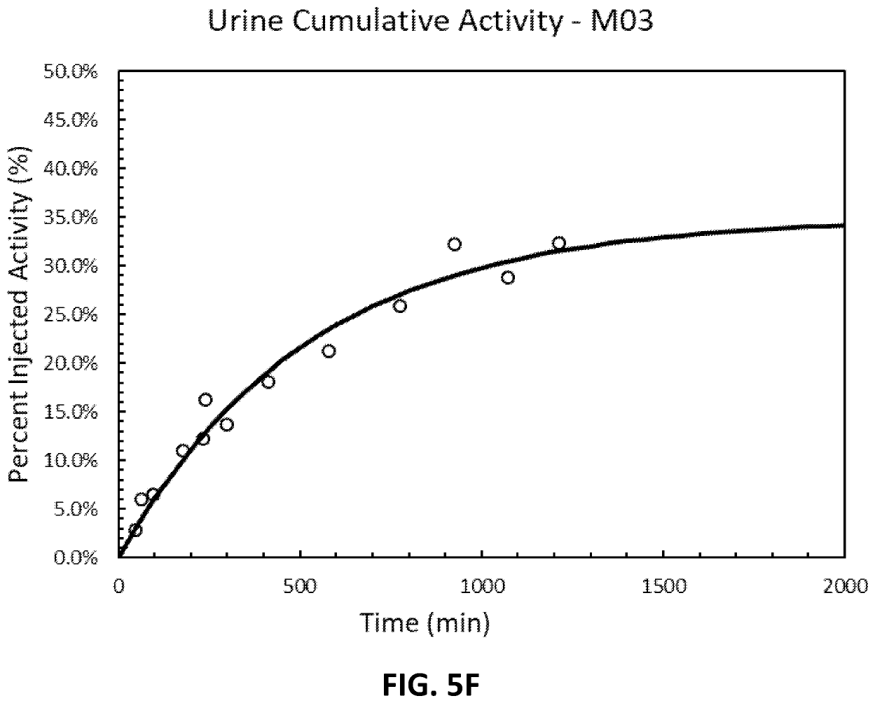

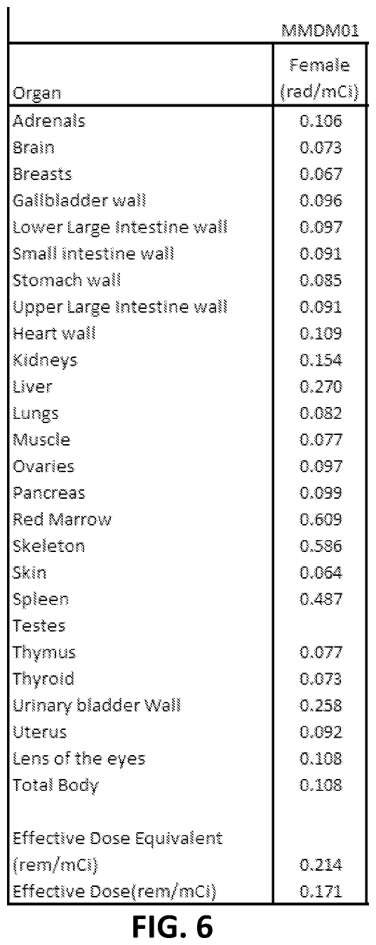

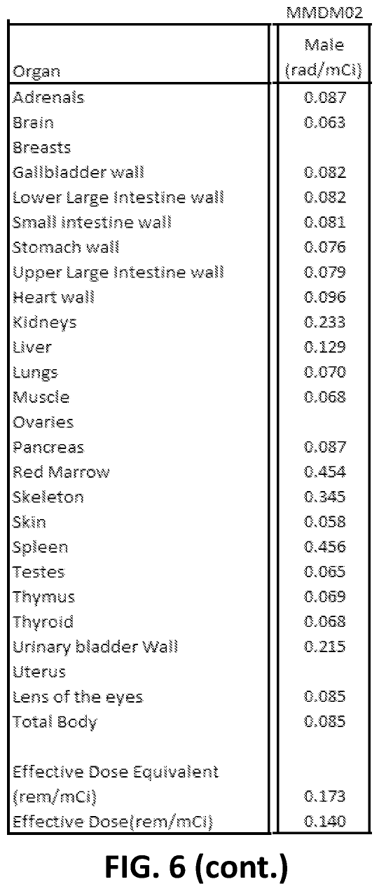

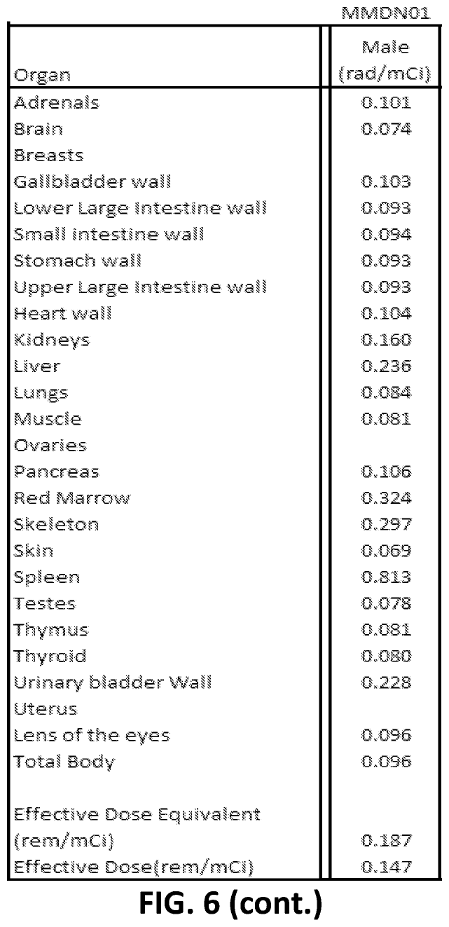

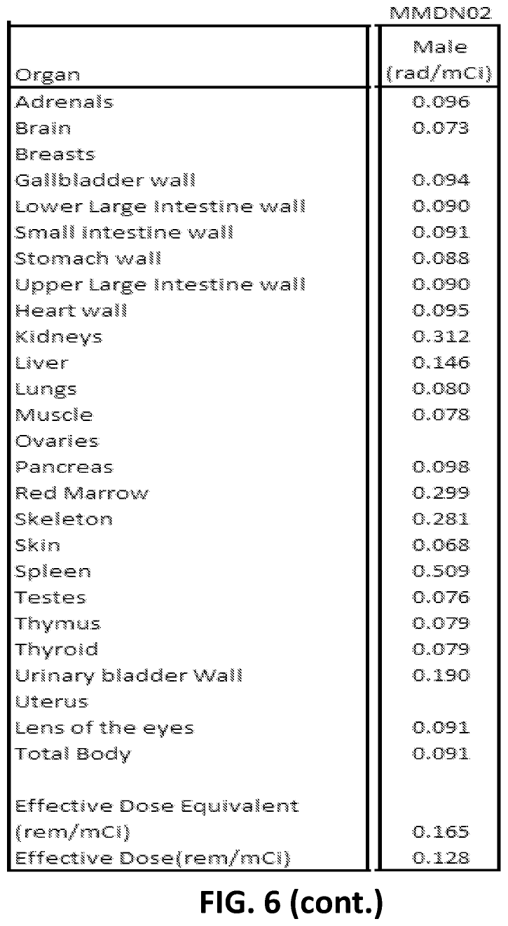

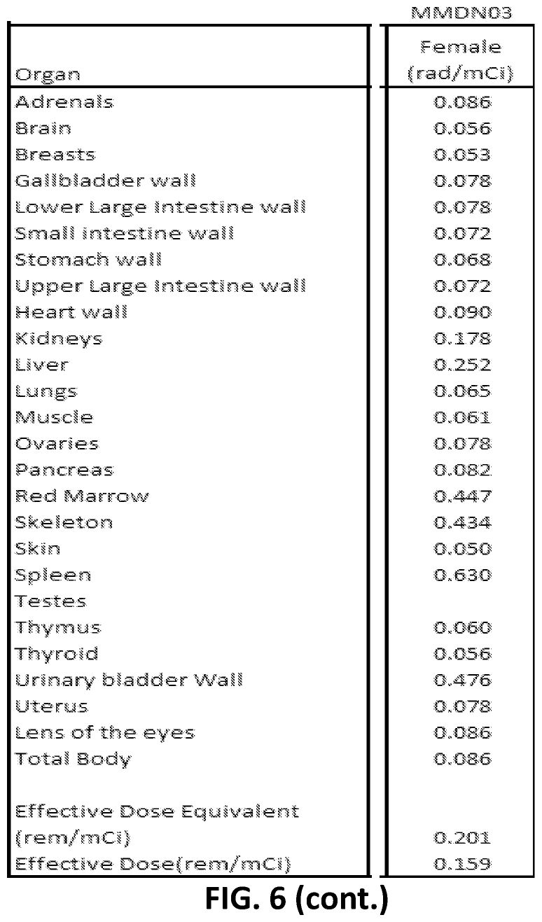

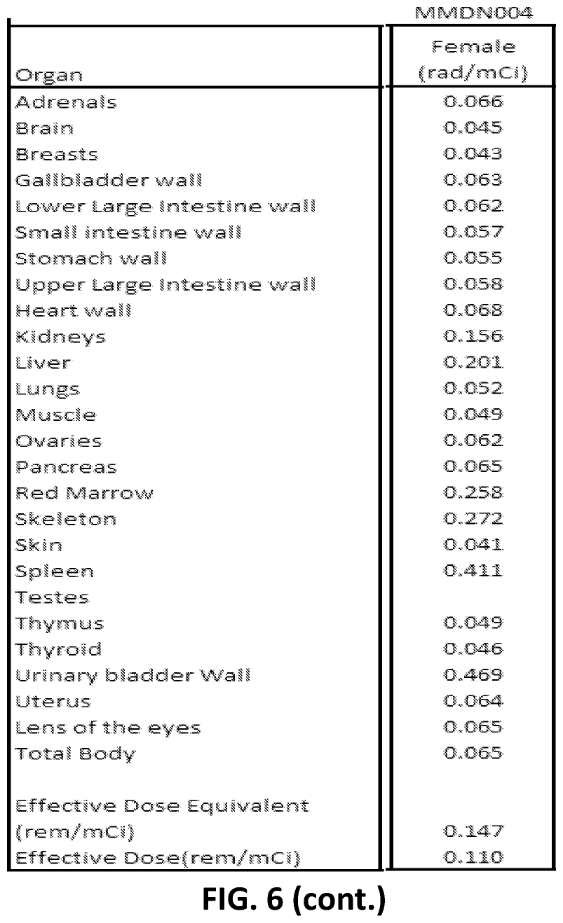

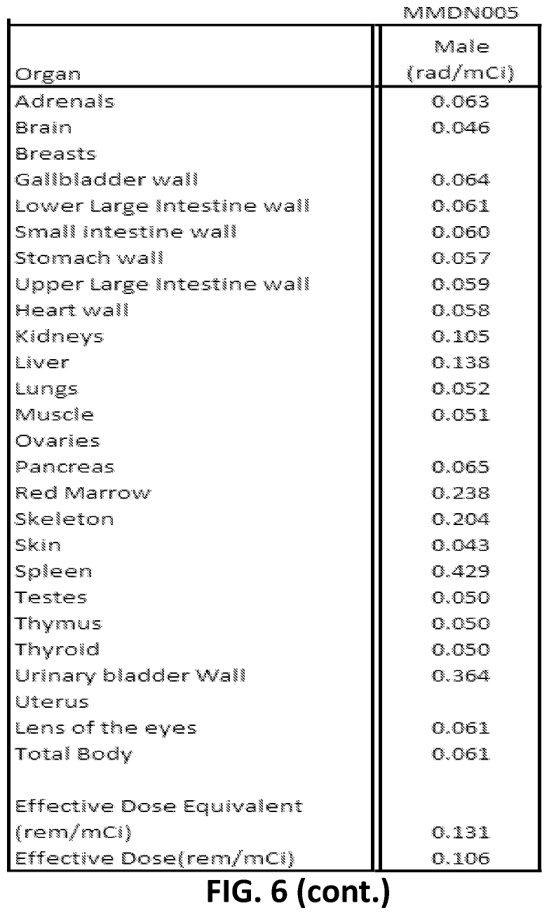

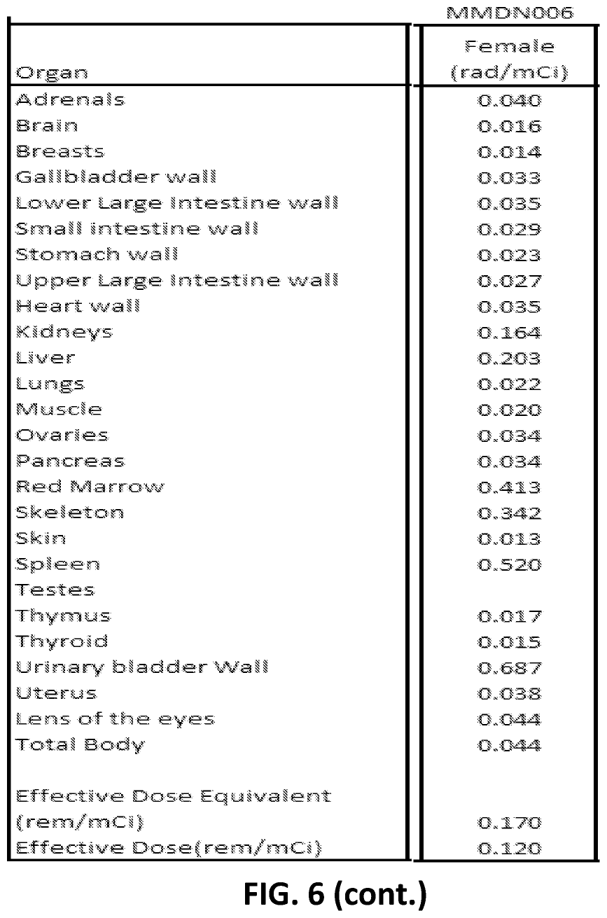

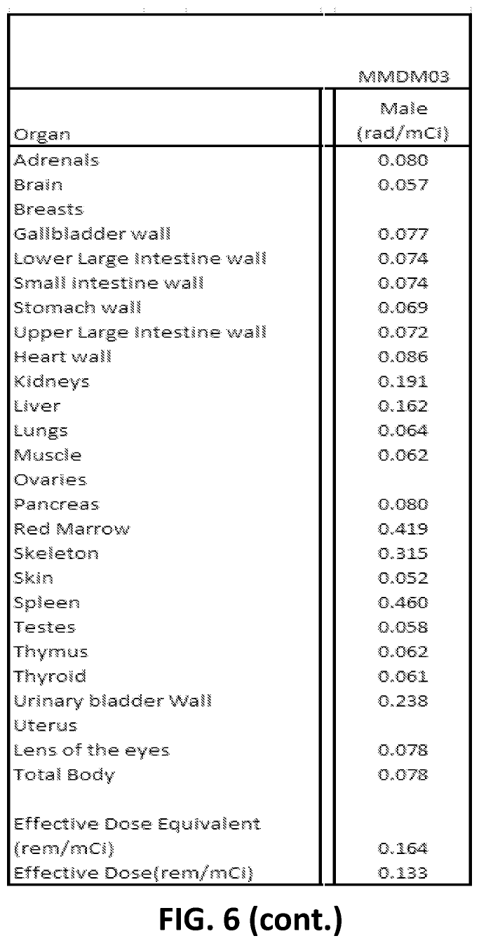

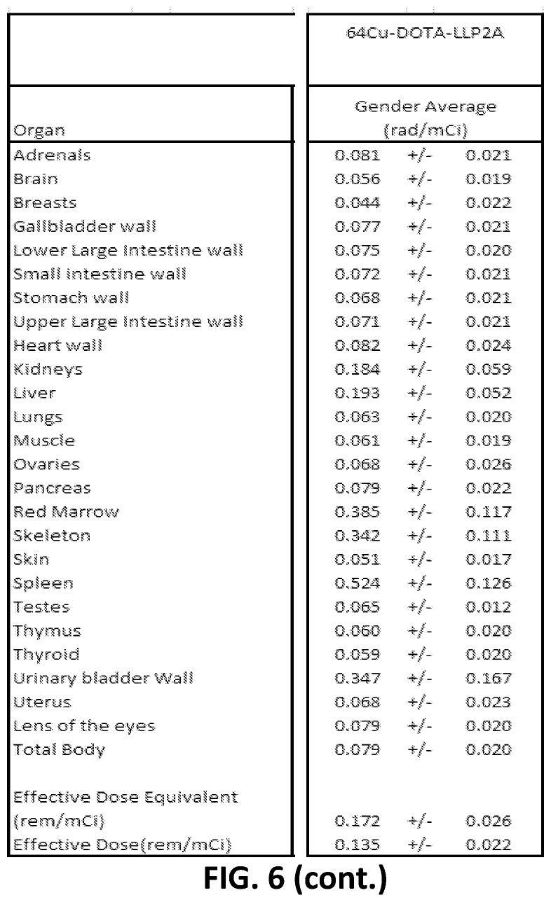

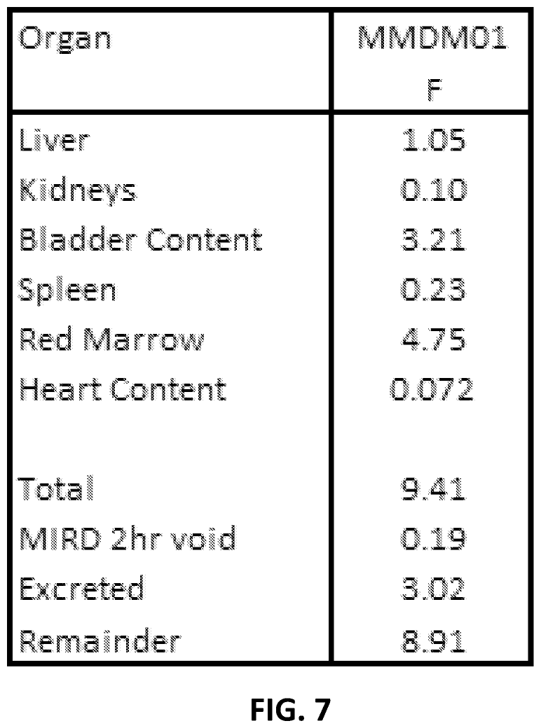

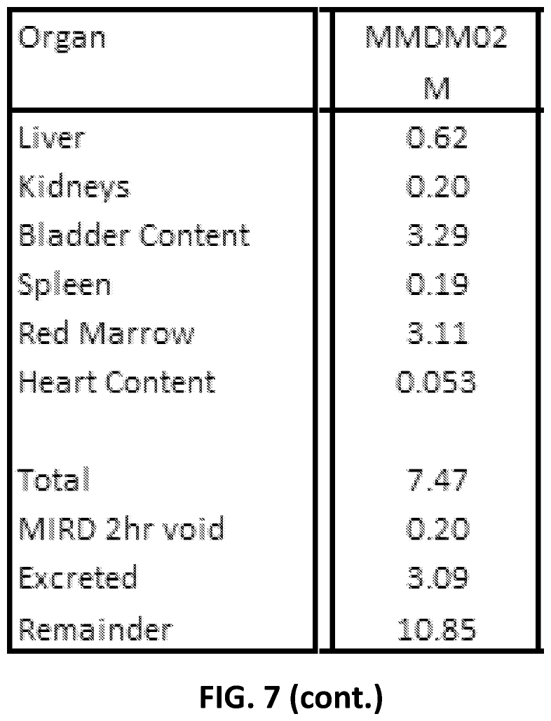

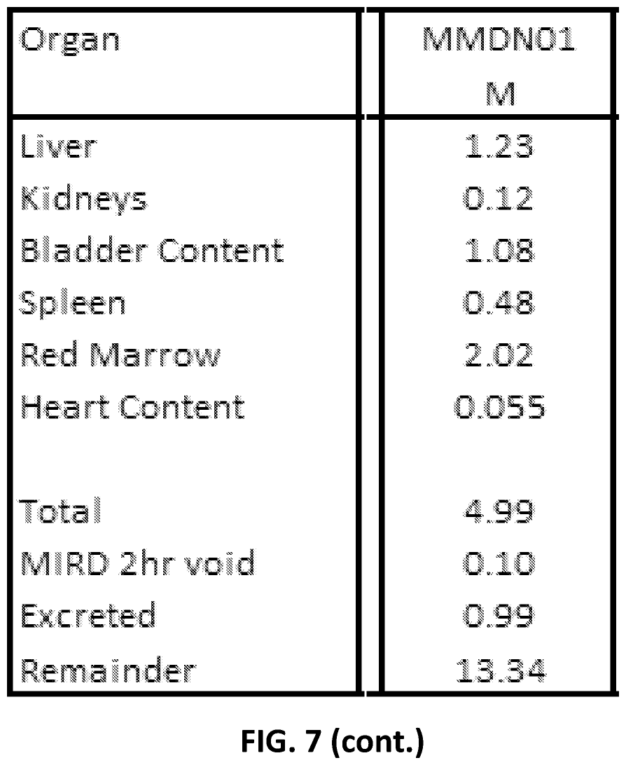

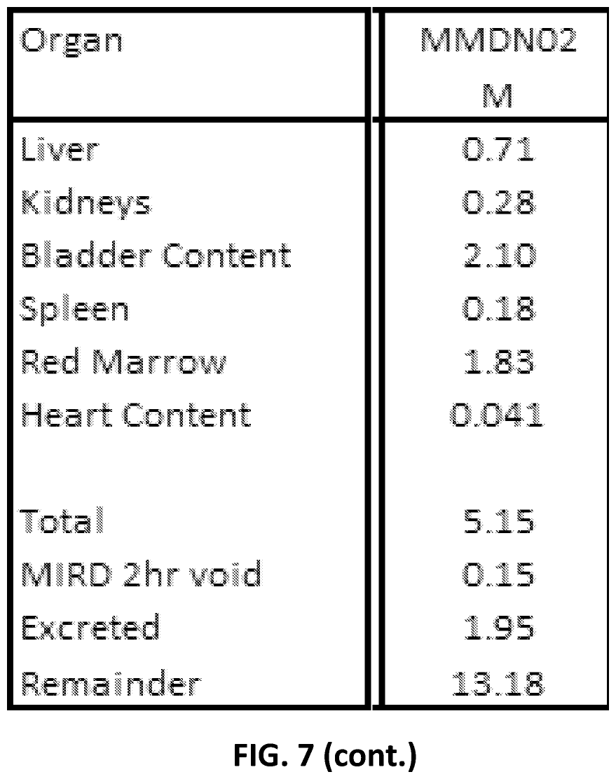

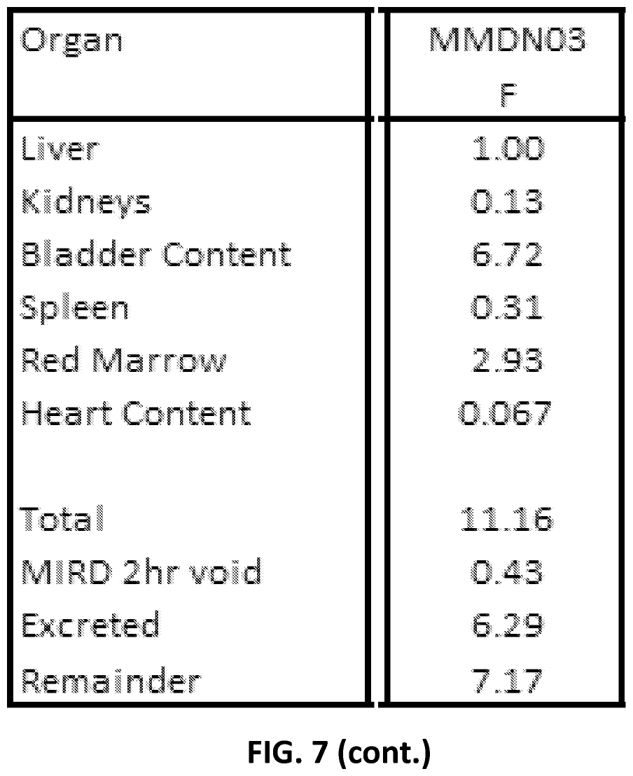

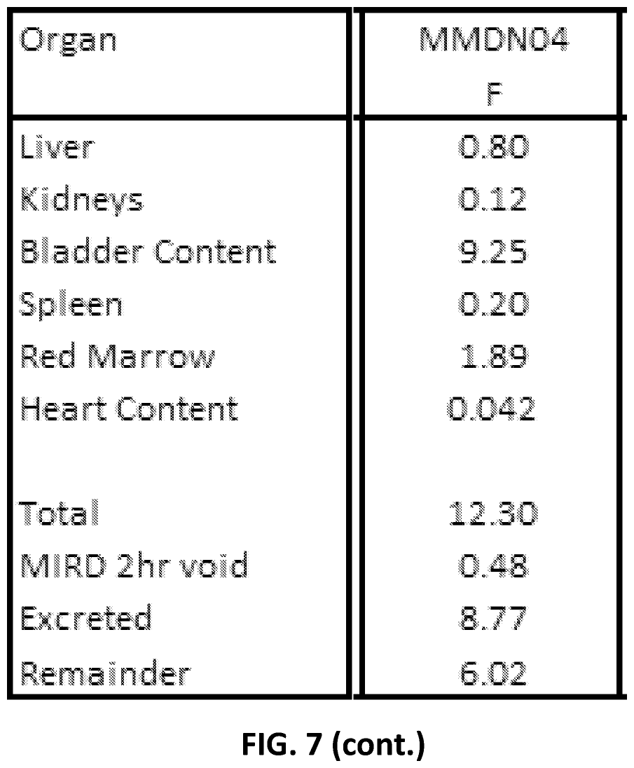

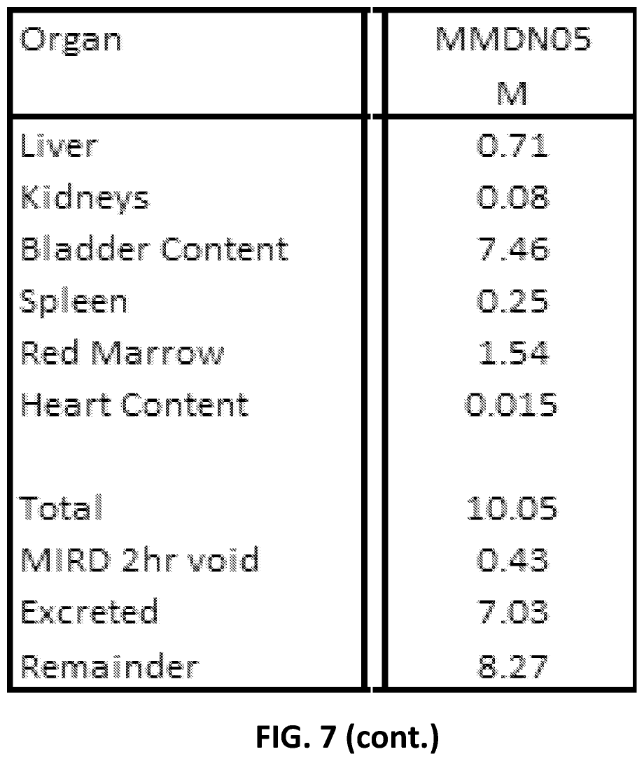

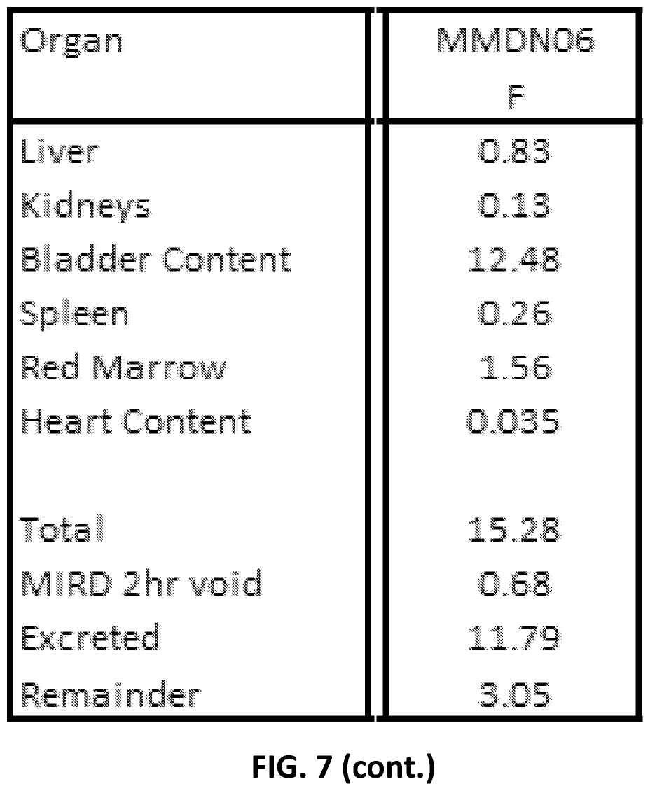

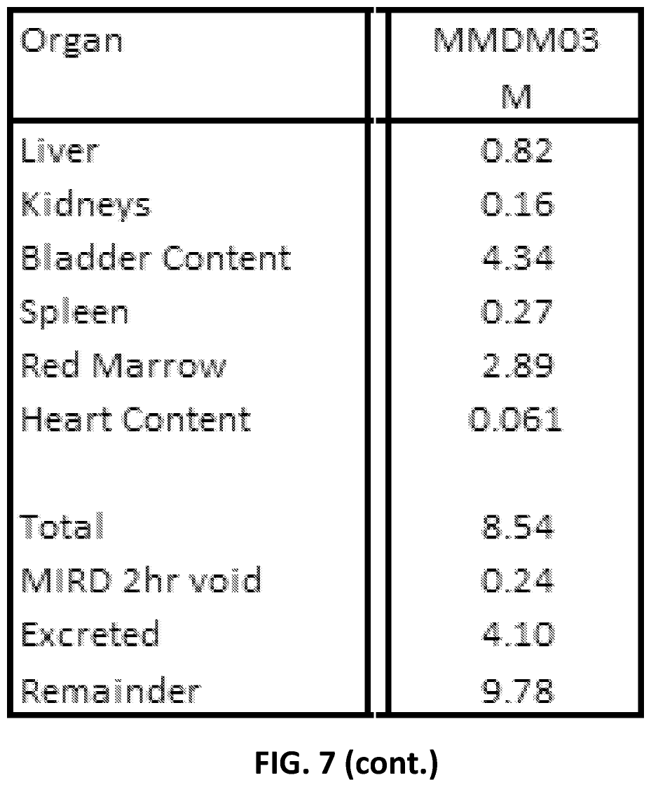

Organ time-integrated activity (TIA) was calculated on a per-patient basis by analytical integration of mono- or dual-exponent fits for the liver, spleen, kidneys, marrow, and blood pool time-activity curves. Aggregated time-activity curves are presented in FIG. 5A-5F. The heart content time-integrated activity was computed from the blood TIA and the total blood volume and heart chamber volume for the adult male or female as defined in ICRP-106. The cumulative urine data (from both imaging and urine collection) were plotted as a function of time and were fitted for each patient with an uptake function of the form (A=Aₒ(1−exp(−A1 t)), where Aₒ is the filing fraction and A1 is related to the filling half-life by the relation ln(2)/A1. The filling fraction and filling half-life were then entered in the MIRD bladder voiding model along with a voiding interval of 2 hours to yield the bladder content TIA. The TIA of the urine excreted activity was then calculated from the TIA of the total urine content minus the bladder content TIA. The remainder of the body TIA was finally computed from the difference between the maximum TIA (Cu-64 half-life of 12.7 hr/ln(2) minus the measured organ TIAs and urine excreted TIA. Organ radiation doses were then computed for the adult male or adult female models using the computer software OLINDA/EXM 1.1. The male, female, and gender average organ radiation dose and effective dose were generated (FIG. 6). The organ residence time was also quantified (FIG. 7).

### Blood Metabolism Study.

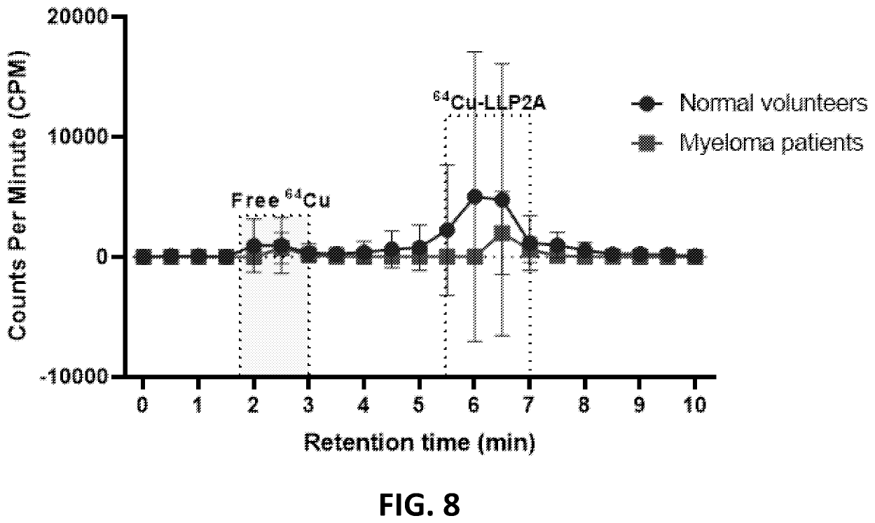

To determine the stability and measure the metabolites of ⁶⁴Cu-LLP2A in human samples, whole blood samples were collected and analyzed by radio-HPLC. Briefly, whole blood samples were collected from all the study participants. Samples were collected at 1-2 h post-injection of the radiotracer in EDTA tubes. Prior to the analysis, the blood samples were centrifuged for 5 min @5000 g, followed by the separation of plasma. The radioactivity was measured in the whole sample (˜1 mL) as well as in post-centrifugation plasma samples. The plasma sample was diluted with Milli-Q water (1:1 ratio) and passed through a 0.22 μm filter before injecting it into the radio-HPLC. 10 μL of the sample was injected into the HPLC and radioactive fractions were collected over a period of 10 min (0.5 mL/30 Sec) (FIG. 8). The radioactivity from the collected HPLC fractions was measured using the gamma counter.

Flow Cytometry Study. Two of the three patients with MM also agreed to provide blood and bone marrow (BM) samples for an institutional banking study of plasma cell dyscrasias. For this study, a sample from one of the MM patients was analyzed. The BMs of CD138 positive and negative populations were sorted by autoMACS pro separator using human CD138 microbeads and indirect human CD34 microbead kit (Miltenyi Biotec, Germany), respectively. Briefly, CD138+cells from the BM of patients were purified by immunomagnetic selection using an autoMACs device. Purified cells were >97% for CD38+CD138+plasma cells. LLP2A-Cy5 synthesis and flow cytometry were performed as previously described. Briefly, cryopreserved human peripheral blood and BM mononuclear cells were thawed, washed in phosphate-buffered saline (PBS), and stained for 15 minutes at room temperature with a LIVE/DEAD Fixable Blue Dead Cell Stain kit (Invitrogen, Carlsbad, CA). Cells were washed in Hanks Balanced Salt Solution (HBSS) containing Ca2+, Mg2+, and 0.1% BSA and incubated for 10 min at room temperature with human Fc Block (BD Biosciences; San Jose, CA). Samples were then incubated for 30 min at room temperature with pre-titrated saturating dilutions of the following fluorochrome-labeled antibodies (clone and source designated in parenthesis): CD45-BUV395 (HI30; BD), CD19-BUV496 (SJ25C1; BD), CD33-BUV737 (P67.6; BD), CD138-BV421 (MI15; BD), CD3-VioBlue (BW264/56; Miltenyi), CD16-BV510 (3G8; BioLegend), CD56-BV711 (NCAM16.2; BD), HLADR-BV785 (L243; BioLegend), CD29-VioBright 515 (REA1060; Miltenyi), CD49d-PE (9F10; BD), and CD11c-PE-Cy7 (B-ly6; BD). Fluorescence minus one controls were used to assess background fluorescence intensity and set gates for negative populations. Samples were analyzed on a ZE5 (Bio-Rad, Hercules, CA) flow cytometer. Single stain compensation controls were obtained using UltraComp eBeads (Thermo Fisher Scientific) and data were analyzed using FCS Express (DeNovo Software, Pasadena, CA).

### Results:

### Cell Binding Assays.

The whole-cell uptake of ⁶⁴Cu-LLP2A at 37° C. in VLA4 expressing 5TGM1 cells was included in quality control (QC) after each radiolabeling (FIG. 3A). The cell uptake in the presence of the blocking agent (unlabeled LLP2A) was significantly reduced (FIG. 3B; % Blocking: 82.42±13.47).

### Animal Toxicity Studies.

There was no test compound (Cu-LLP2A) related effect on body weight observed following one I.V. dose administration (FIGS. 4A and B). There were no definitive Cu-LLP2A-related changes in clinical observations, pathology, clinical chemistry, and hematology parameters (FIGS. 4C and D). Some minor differences in individual or group mean hematology and clinical chemistry values in male/female mice given Cu-LLP2A compared to control mice were insignificant, within the observed normal range for mice of this strain and age, were single in occurrence, or were not dose-dependent, and, therefore, were not considered Cu-LLP2A related. There were no macroscopic observations in animals administered Cu-LLP2A. The only gross lesions documented were in control mice that consisted of a focal, unilateral ovarian cyst, and an abnormally shaped left kidney. Based on the normal clinical observations coupled with no definitive Cu-LLP2A-related effects on body weights, clinical pathology parameters, organ weight values, and microscopic findings, a single I.V. injection of 0.0103 mg/mouse of Cu-LLP2A was well tolerated in male and female CD-1 IGS mice and was considered NOEL.

### Clinical Studies

Six healthy volunteers and three subjects with confirmed multiple myeloma diagnoses (median 30 years, range 25 to 83 years) participated in the study. The subjects received a median dose of 352.24 MBq (range 247 to 433 MBq) of ⁶⁴Cu-LLP2A. The targeted administered amount of VLA4 targeted radiotracer, LLP2A was 15 μg (based on NOEL) and the mean radioactivity administered per patient was 352.24 MBq (9.5 μg) with a maximum specific activity of 572.7 MBq/nmol (1000 μCi/μg). The radiochemical purity of >90% was confirmed by radio-HPLC.

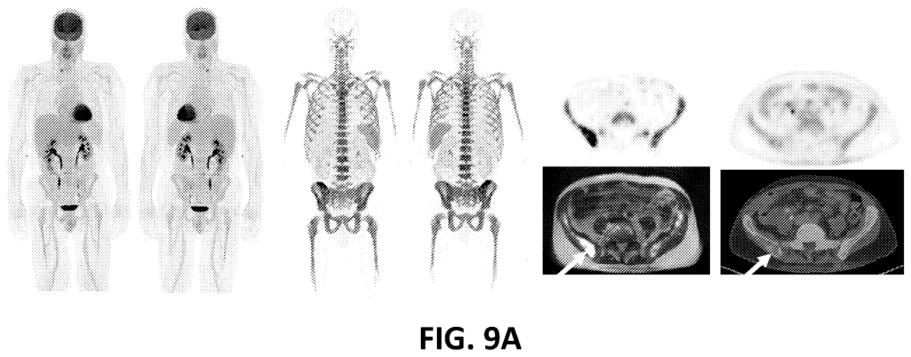

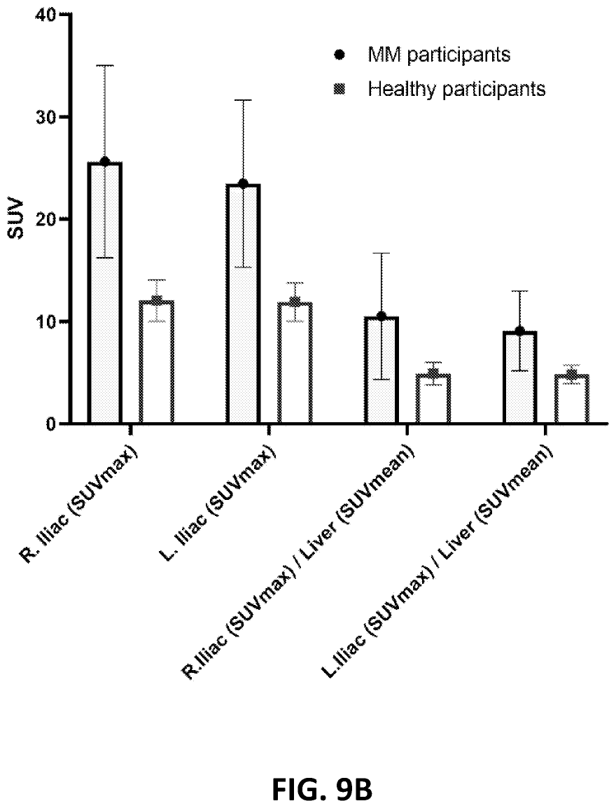

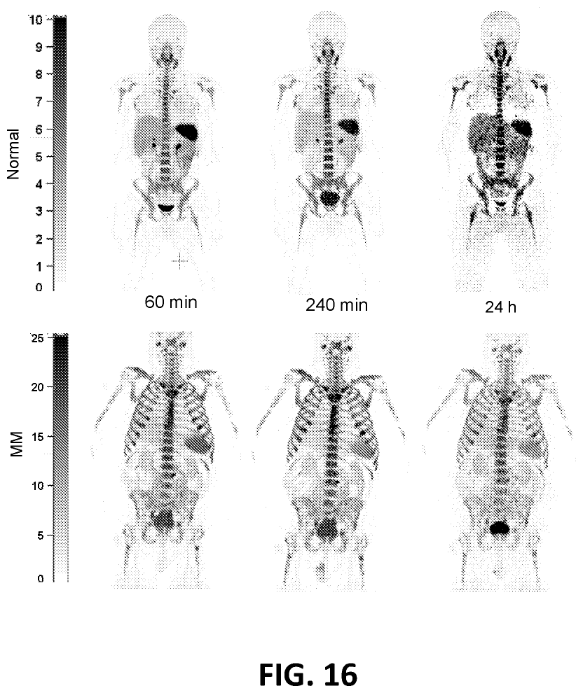

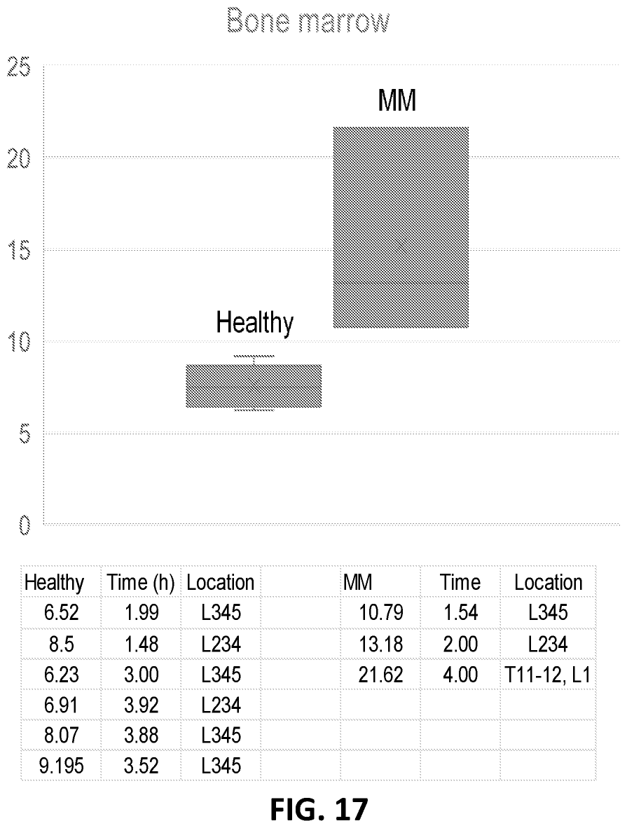

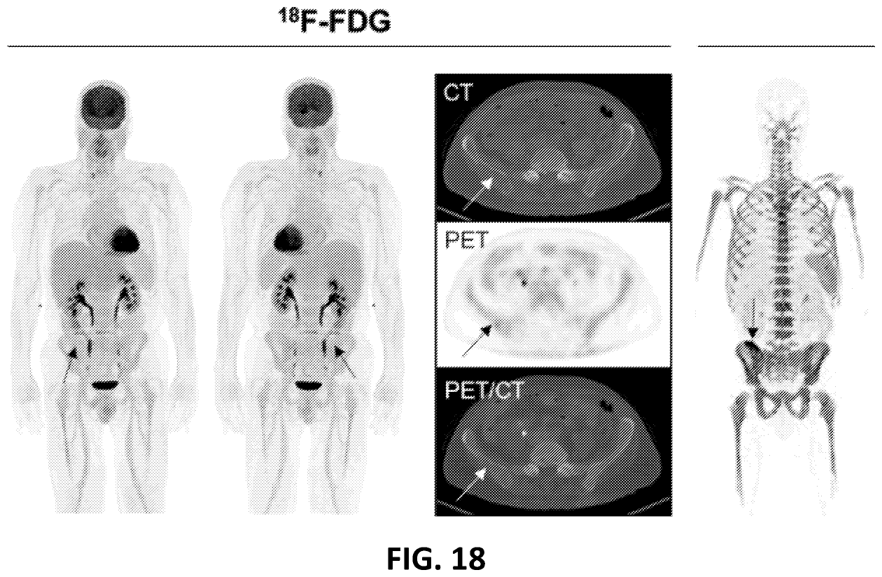

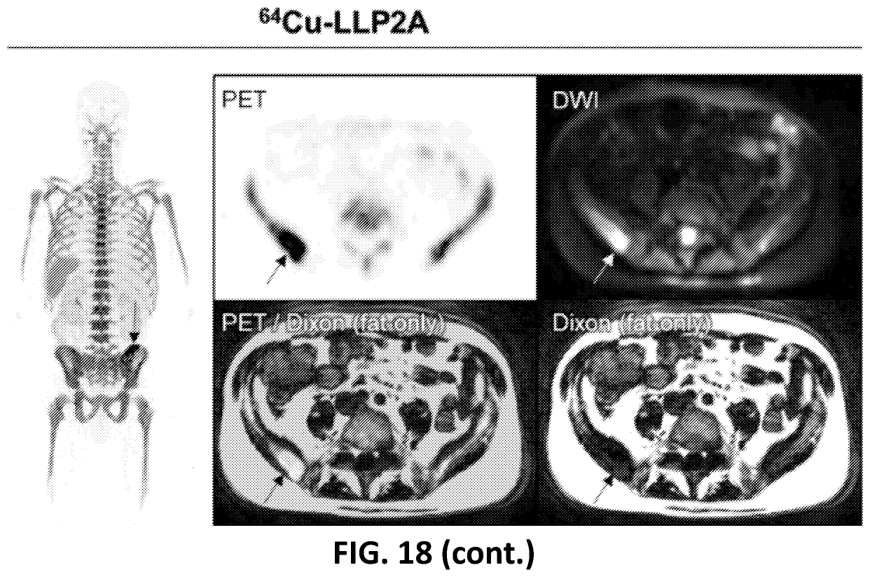

Based on qualitative analysis, the best quality images were performed between 1-5 hrs post-injection, the images that were performed the next day, typically around 24 hrs were noisy (FIG. 9A and FIG. 16). The mean and standard deviation for SUVₘₐₓ of iliac bones in healthy volunteers were 12.05±2.0 while it was 25.62±9.38 for multiple myeloma patients (FIG. 9B). The bone marrow SUVₘₐₓ was measured as the average of lumbar vertebra 3, 4 and 5 for most patients (FIG. 17). Two subjects had a new diagnosis of multiple myeloma by bone marrow biopsy and elevated immunoglobulins; both had negative FDG-PET/CT for diffuse or focal areas of increased uptake; however, one had a lytic non-FDG-avid lesion in the right iliac bone. This MM participant's scan (MMDM02) demonstrated diffuse moderate T1-hypointensity of the marrow in the spine and pelvis, similar to intervertebral discs and skeletal muscle, which indicates diffuse marrow infiltration (FIG. 18). The remaining patient had recurrent multiple myeloma with increasing M spike on a regimen of elotuzumab, his FDG-PET/CT and bone survey were negative. Additionally, the MR scan for MMDM01 showed heterogeneous mild T1-hypointensity of marrow in the spine and pelvis but not as T1-hypointense as the intervertebral discs and skeletal muscle, which could be contributed to red marrow; not sufficient to call diffuse marrow infiltration on MRI. ⁶⁴Cu-LLP2A-PET demonstrated an overall diffuse moderately increased uptake throughout the bone marrow in all three patients. There was focally intense uptake in the iliac bone lytic lesion of the 2^{nd} patient (FIG. 9A).

### Safety Evaluation

The mean and SD of the administered mass of ⁶⁴Cu-LLP2A was 9.52±1.33 μg (range, 6.9-11.7 μg). The mean administered activity was 352.24±49.19 MBq (range, 175.5 MBq). There were no adverse or clinically detectable pharmacologic effects in any of the subjects. No significant changes in vital signs or the results of laboratory studies or electrocardiograms were observed.

### Blood Metabolism Study Shows Stable Compound at 2 h.

Blood metabolism studies performed with the blood samples primarily showed two peaks on the radio-HPLC chromatogram; one at a retention time of 2-3 min, and the other at a retention time of 5.5-7 min. The trend of metabolite samples on the analytical radio-HPLC matched the chromatogram of ⁶⁴Cu-LLP2A. The data showed only minimal degradation of the radiotracer in vivo.

### Flow Cytometry Study

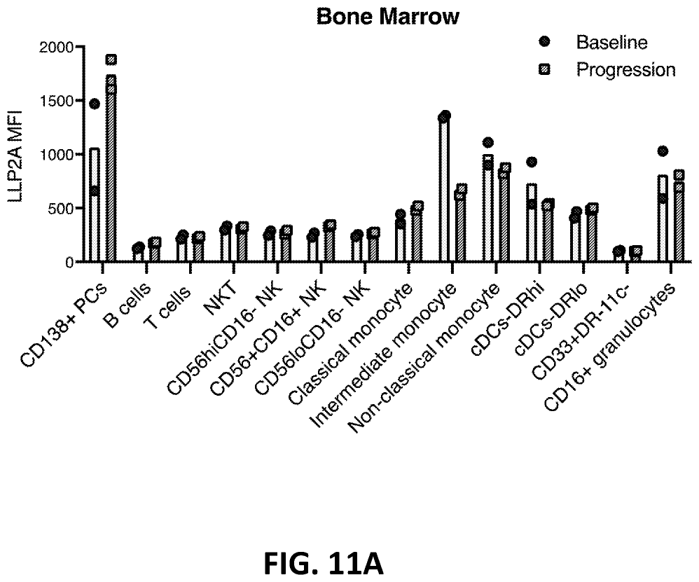

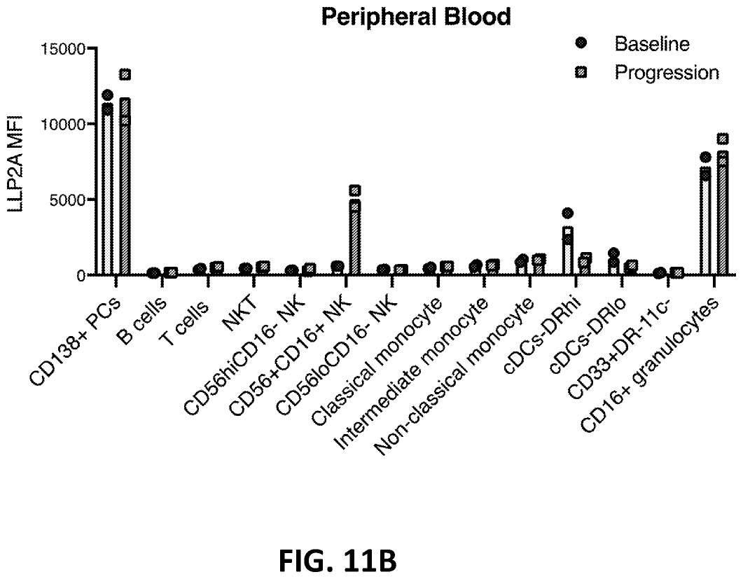

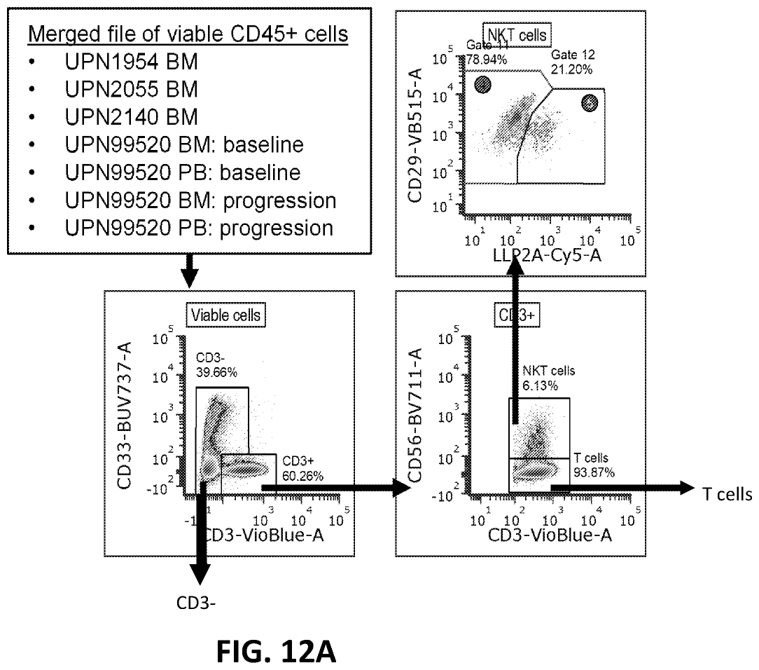

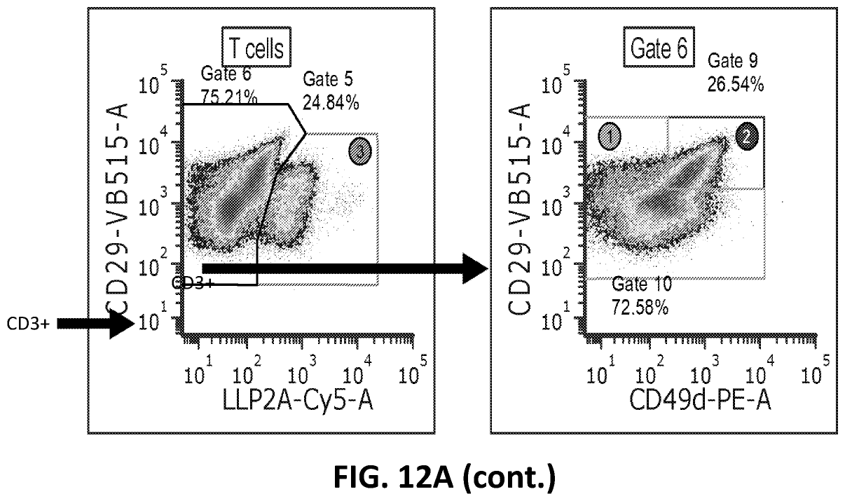

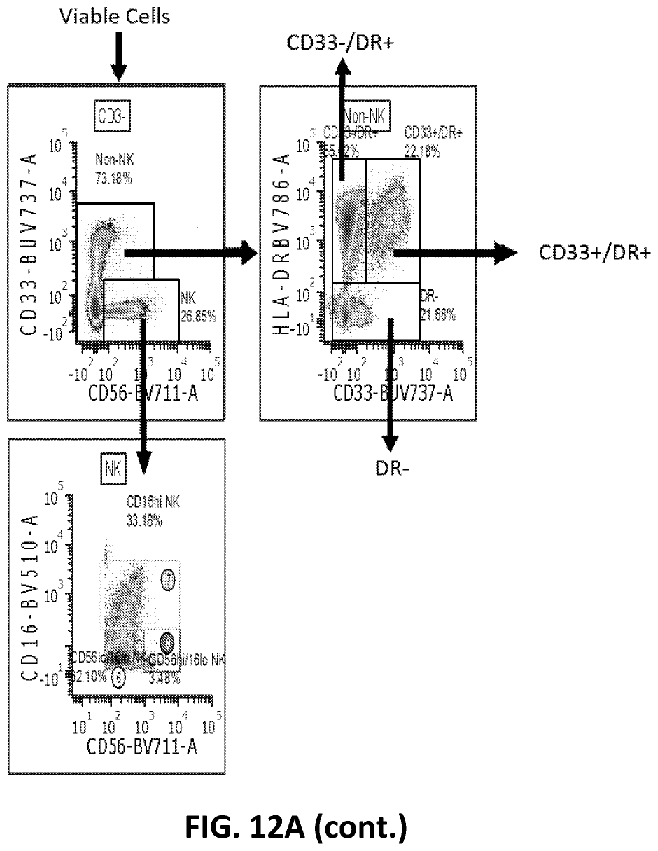

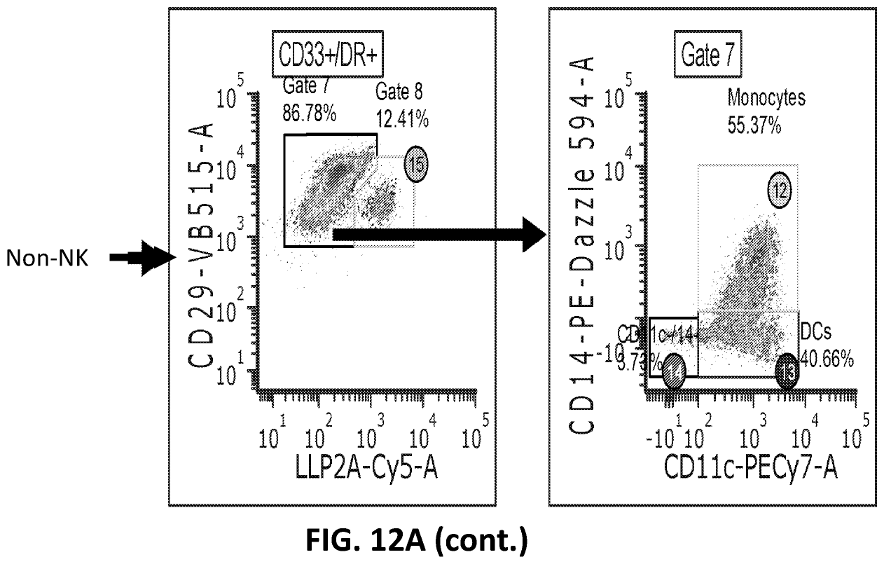

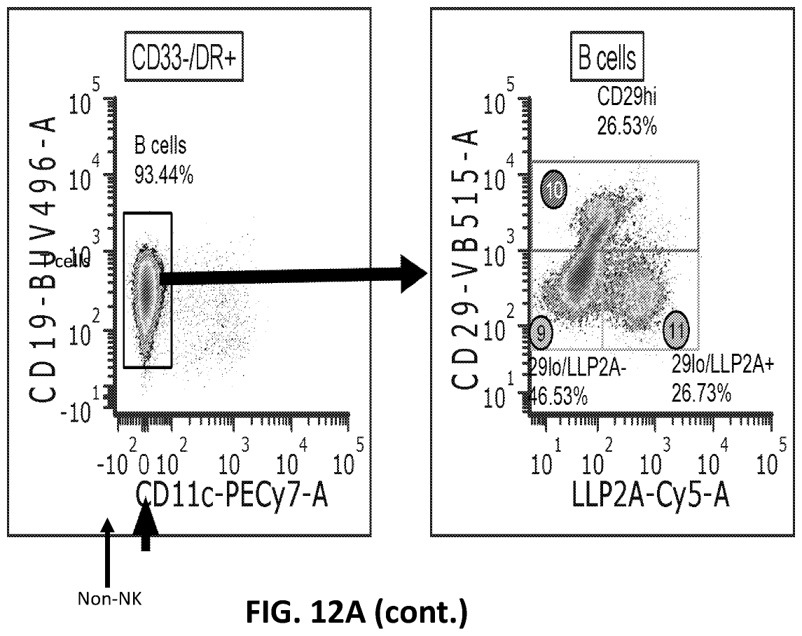

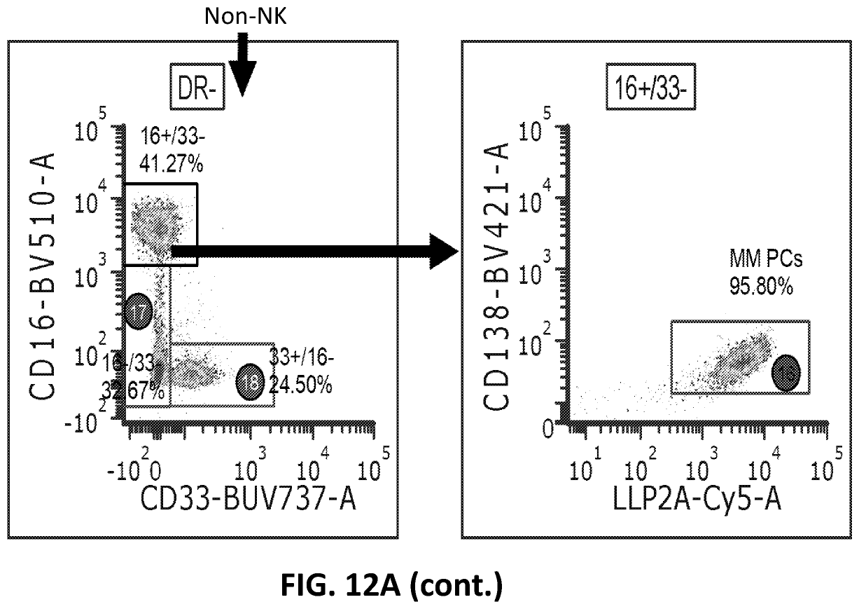

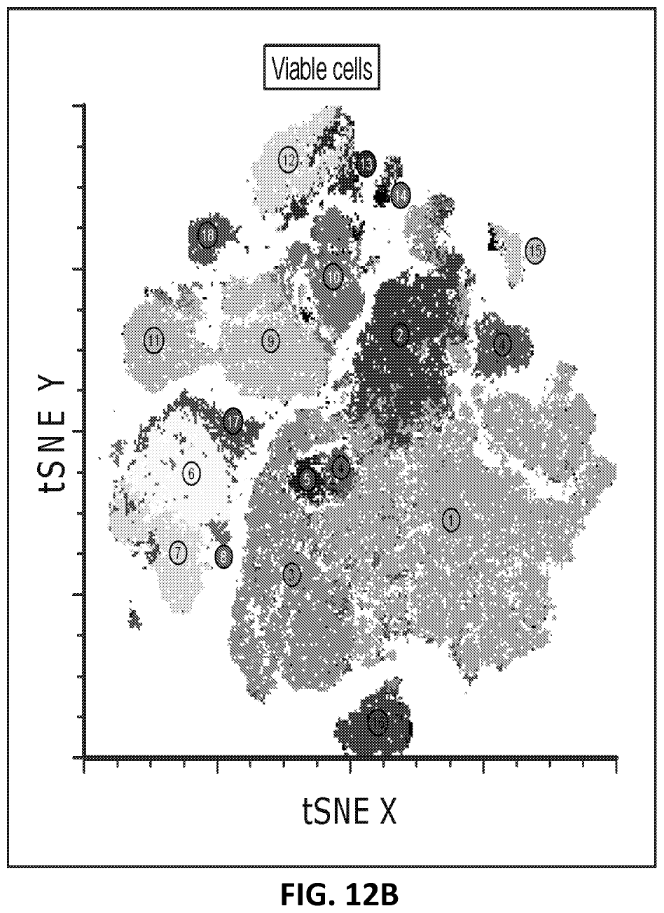

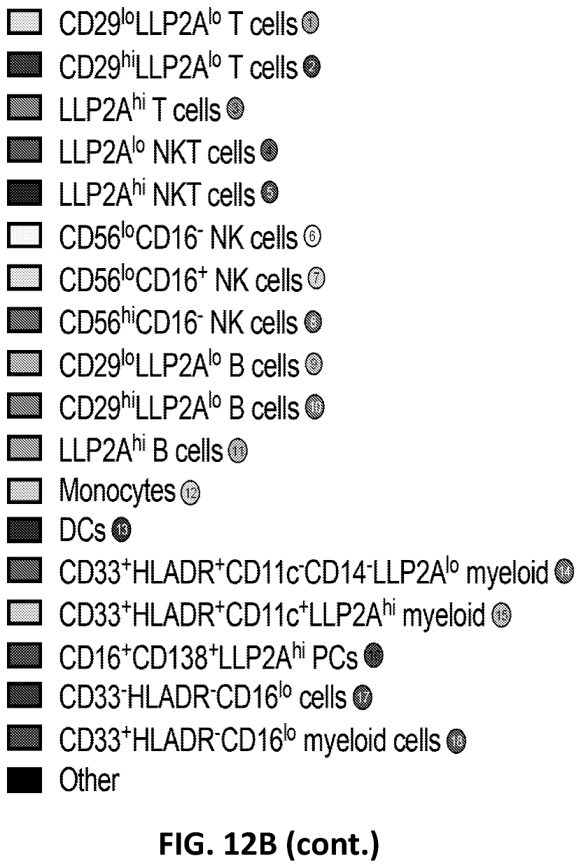

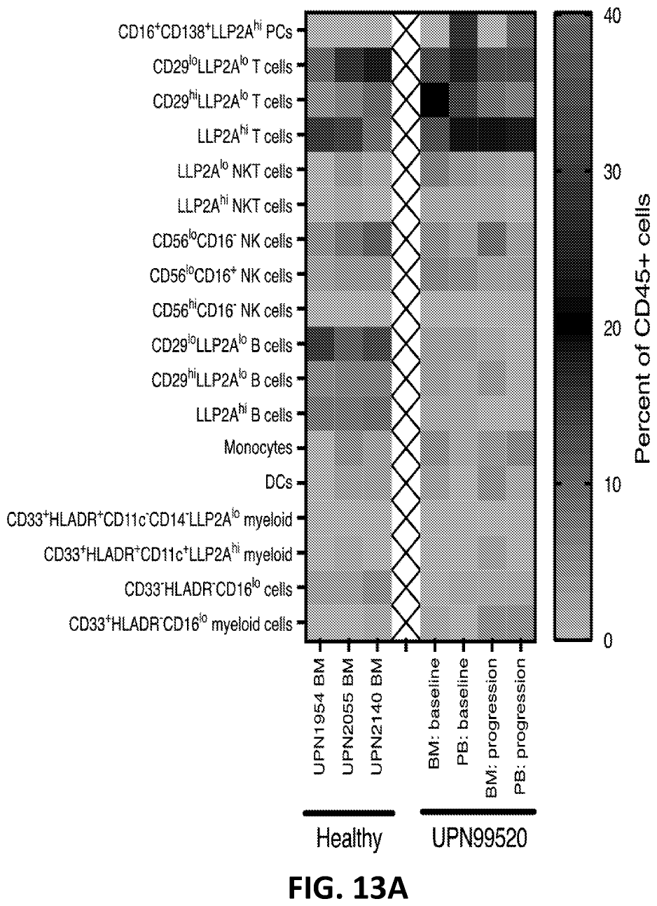

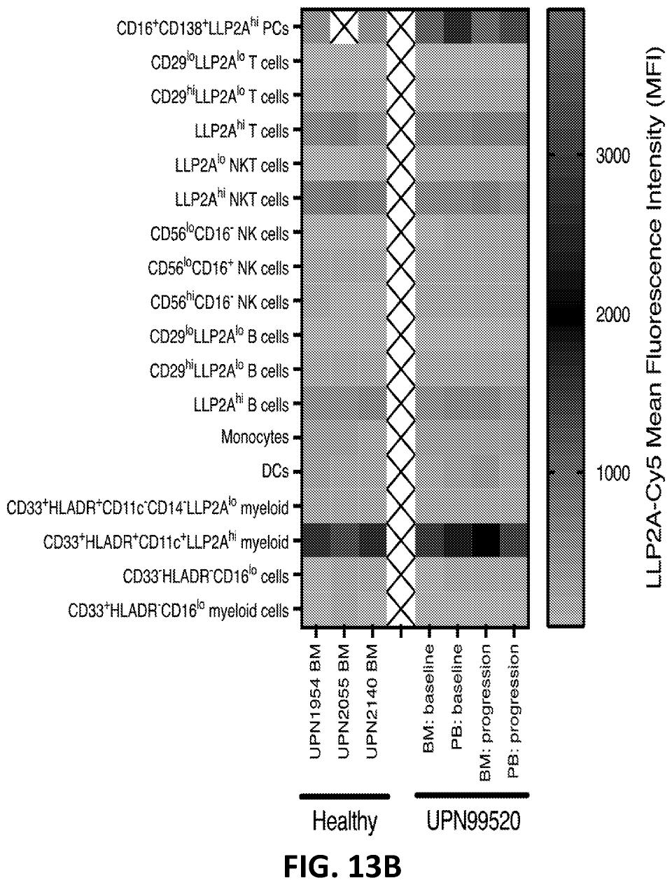

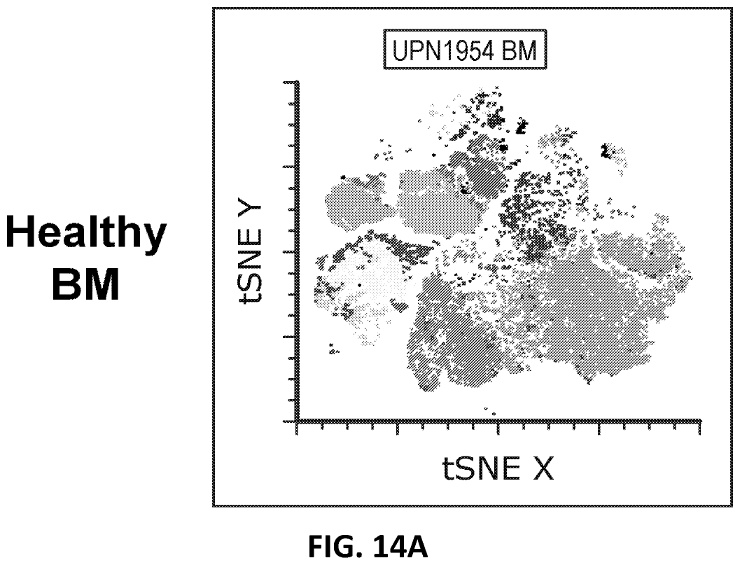

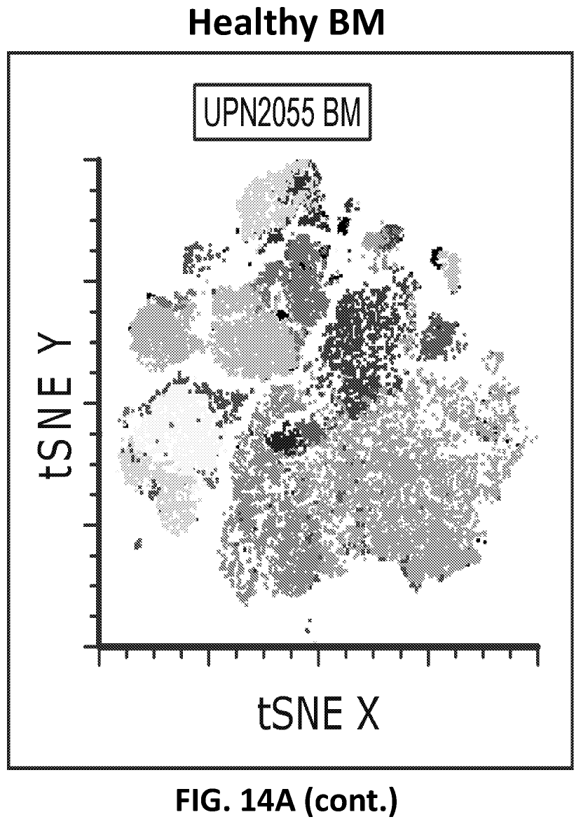

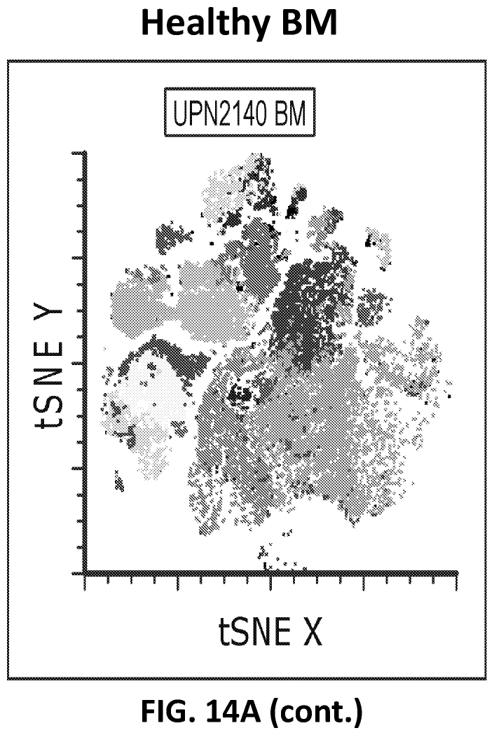

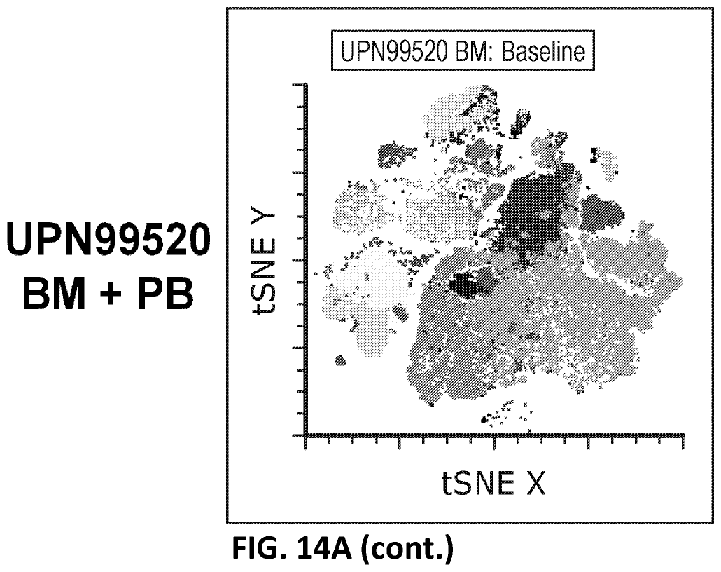

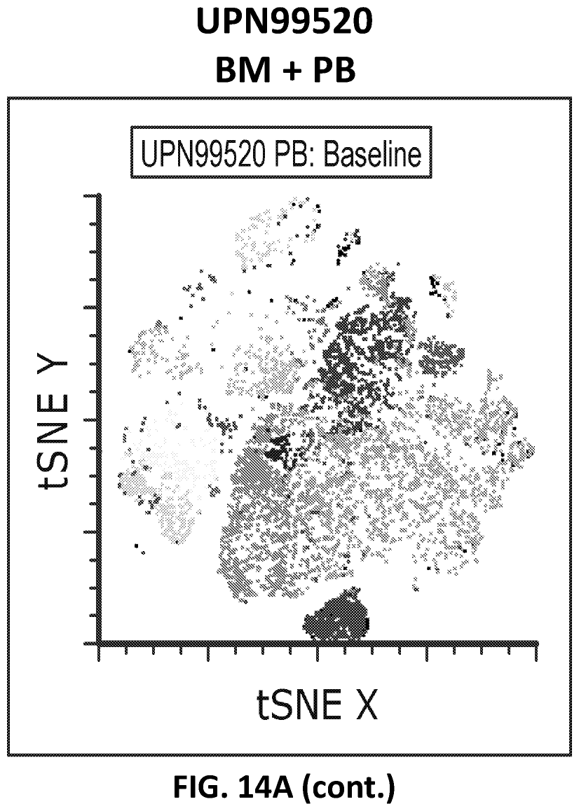

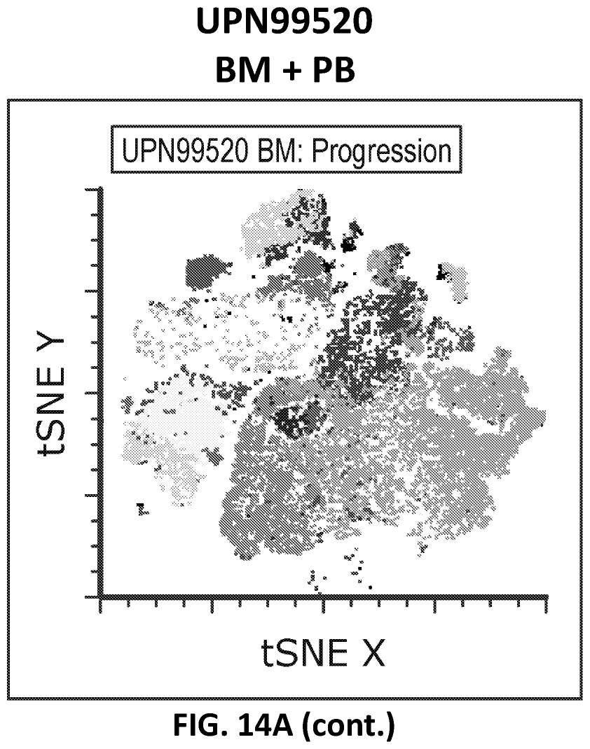

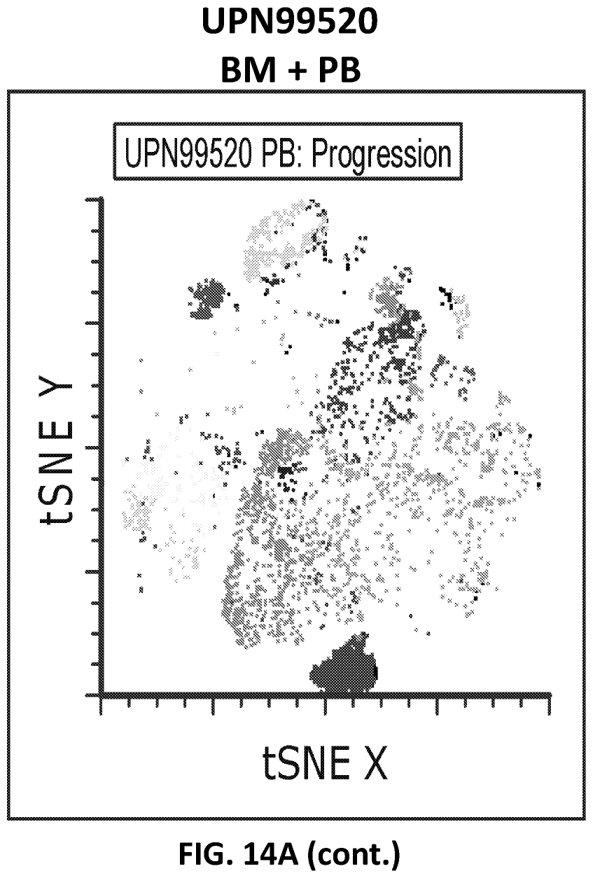

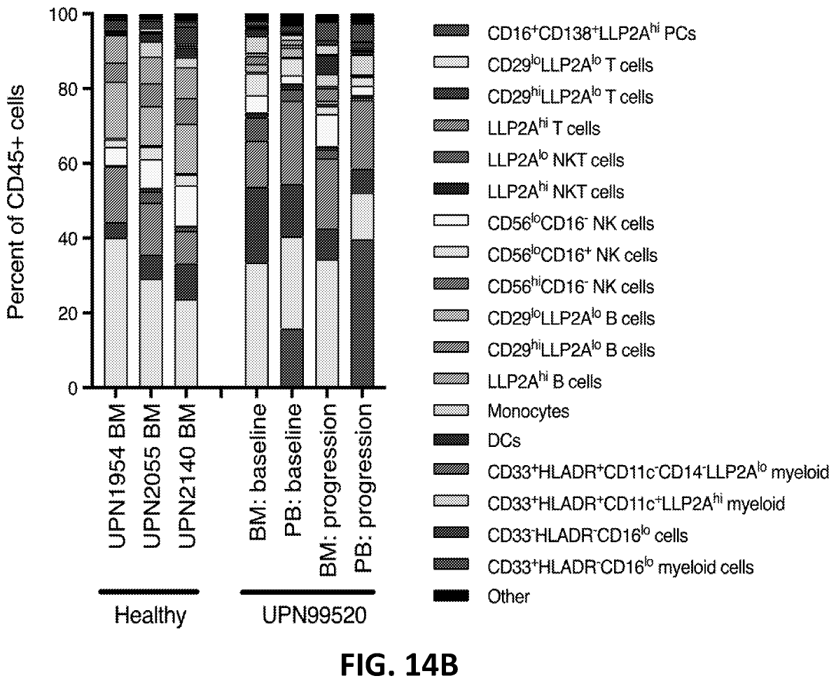

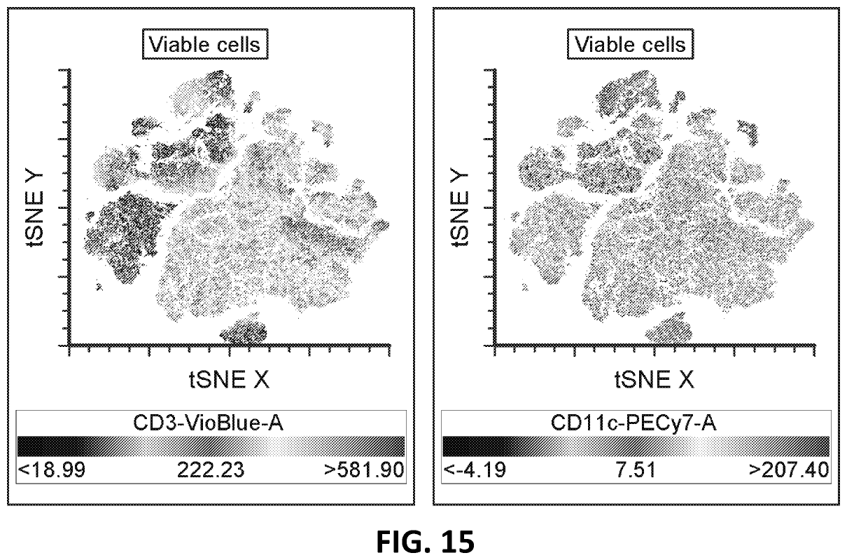

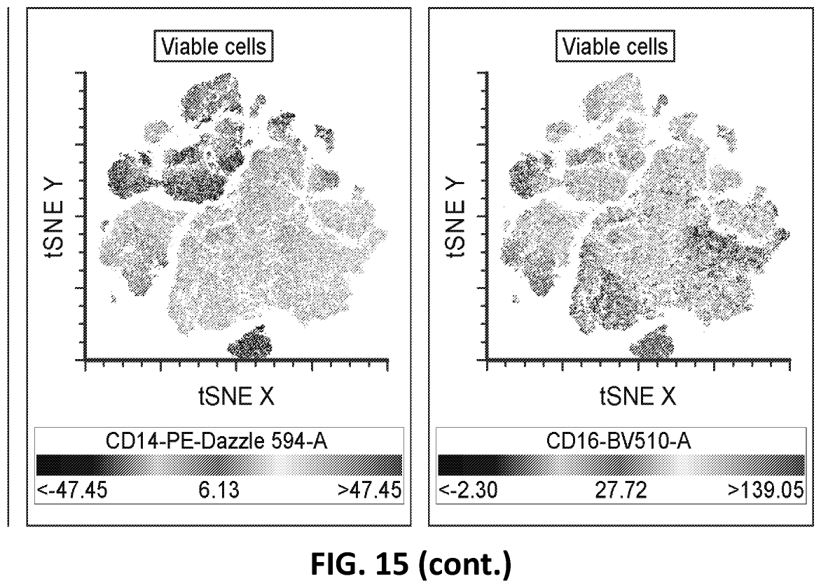

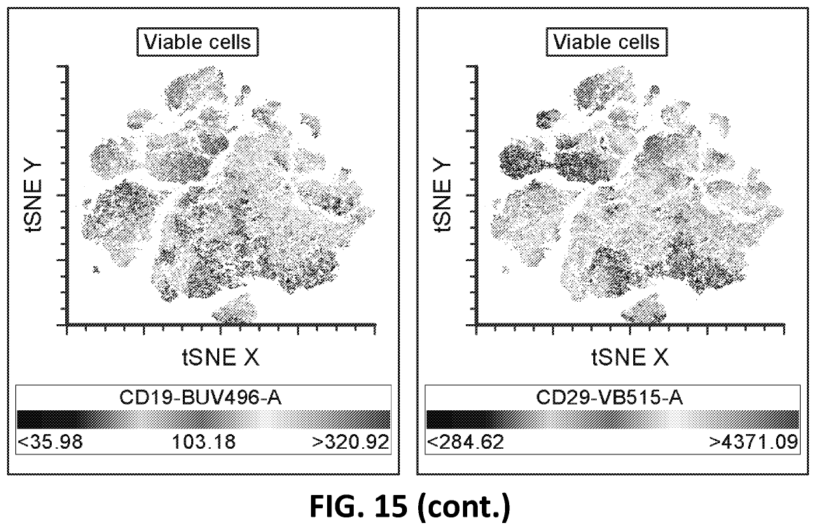

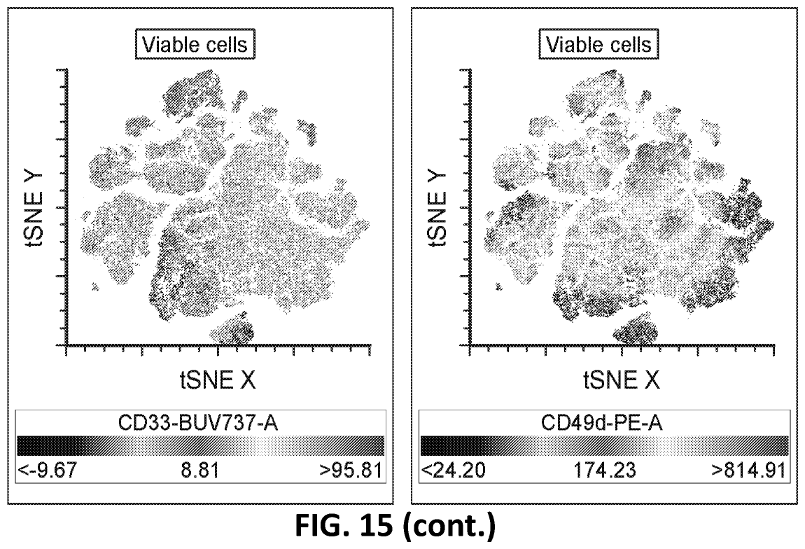

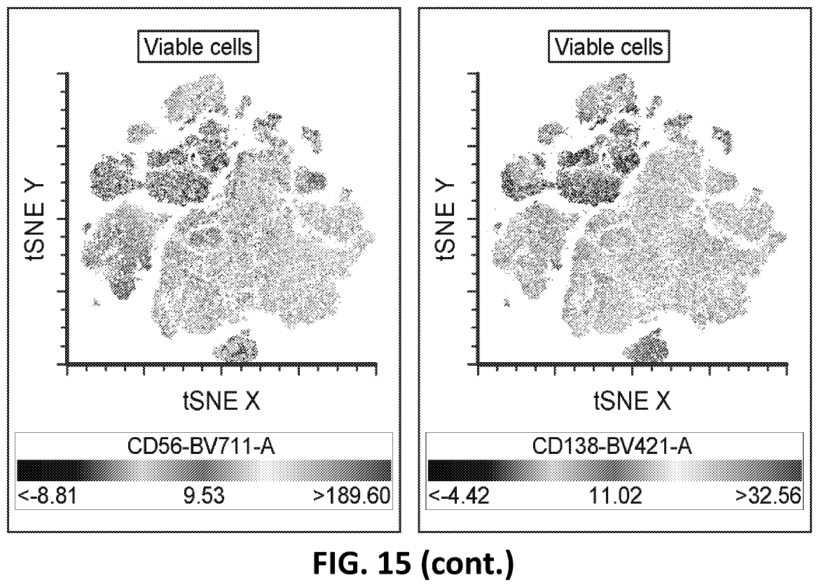

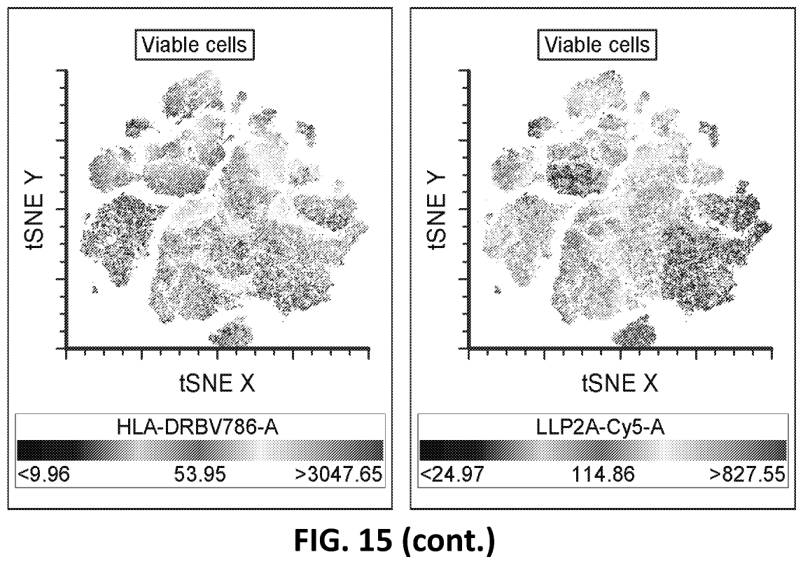

The expression of activated VLA4 on hematopoietic cell populations found within the BM and/or peripheral blood of three healthy donors (UPNs 1954, 2055, 2140) and a patient with MM (MMDM02) (baseline and after disease progression four months later) was examined by flow cytometry using LLP2A-Cy5. Using a 14-color flow cytometry panel, we identified eighteen different hematopoietic cell populations within these seven samples (FIG. 12). The bone marrow mononuclear cells exhibited similar cellular distributions with the exception of fewer mature B cells in the patient with MM compared to the healthy control (FIG. 11A, FIG. 13A, and FIG. 14). In contrast, the peripheral blood samples from the patient with MM were enriched for CD138+ plasma cells expressing high levels of CD16 and activated VLA-4, as measured with LLP2A-Cy5 (FIG. 11B, FIG. 13B, and FIG. 15). As we previously described, subsets of B cells, T cells, NKT cells, and myeloid cells expressing activated VLA-4 (LLP2A^{hi}) were identified in both the healthy controls and MM samples.

### Example 2

To characterize the modulation of molar activity for achieving high contrast in the bone marrow, the following experiments were conducted.

Preclinical ⁶⁴Cu-LLP2A Imaging with Different Specific Activities

LLP2A was radiolabeled with copper-64 (⁶⁴Cu) at two different molar activities of 45.9 MBq/nmol (800 μCi/μg) and 14.36 Mbq/nmol (250 μCi/μg). The radiochemical yield and purity were evaluated by radio-HPLC.

### Multiple Myeloma Mouse Models

The murine myeloma cell line, 5TGM-GFP, was obtained from Prof. Katherine N. Weilbaecher (Department of Medicine, Oncology Division, Washington University School of Medicine, St. Louis). These cells were used to develop the models in KaLwRij (male and female) mice. 5TGM-GFP (1e⁶ cells in 100 μL) were injected subcutaneously on the right flank of the mice and via the tail vein to establish subcutaneous and disseminated myeloma mouse models respectively. Tumors were allowed to grow for 3-4 weeks.

### Small Animal PET Imaging

⁶⁴Cu-LLP2A imaging was performed in KaLwRij mice with no-tumor control mice and mice bearing 5TGM1 subcutaneous and disseminated myeloma tumors, respectively. Prior to small animal PET/CT imaging, the no-tumor, subcutaneous, and disseminated tumor-bearing mice were randomly divided into two groups—the first group received ⁶⁴Cu-LLP2A with lower molar activity, i.e., 14.36 MBq/nmol and the second group received the radiotracer labeled at the higher molar activity of 45.9 MBq/nmol. Mice in each group received about 2-3 MBq of ⁶⁴Cu-LLP2A via tail vein injection and PET/CT was done at 4h post-injection of the radiotracer. Tissue biodistribution studies were conducted in no-tumor and disseminated tumor mice, post imaging to evaluate the uptake of ⁶⁴Cu-LLP2A with different specific activities in healthy and 5TGM1-bearing KaLwRij mice.

Small Animal [⁶⁴Cu]Cu-LLP2A/PET Imaging with Two Different (High and Low) Specific Activities

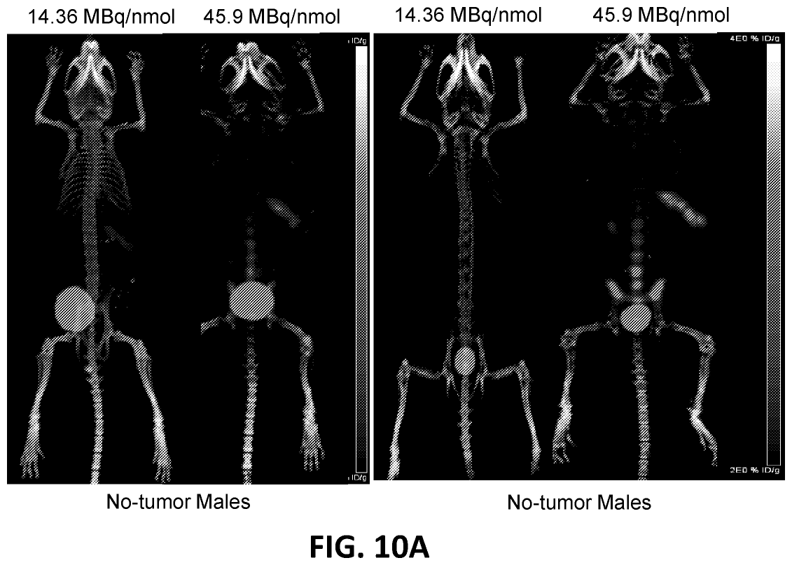

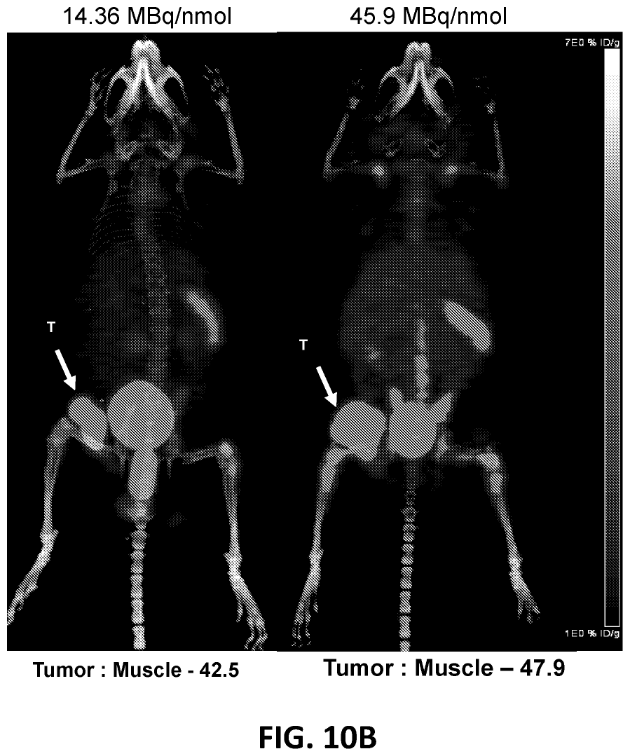

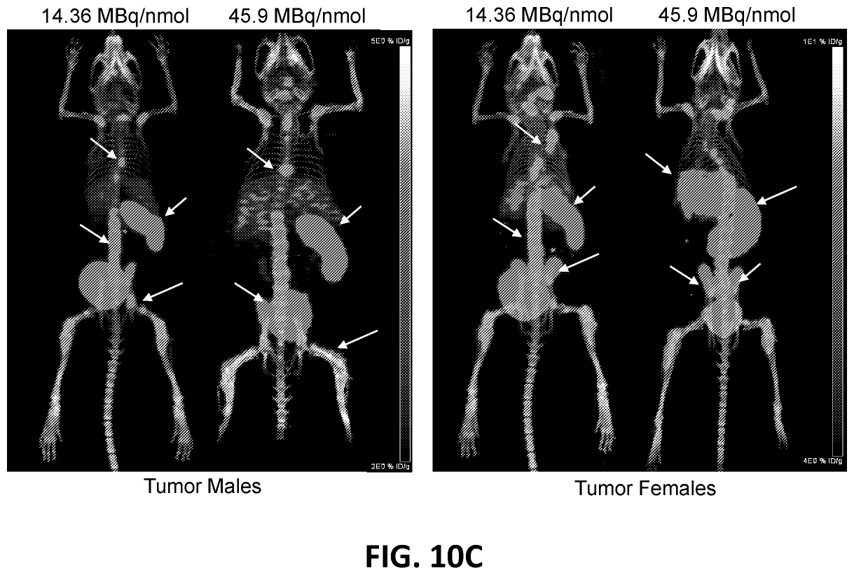

In the healthy cohort, both male and female mice imaged with a lower specific activity of ⁶⁴Cu-LLP2A (14.36 MBq/nmol; 0.28 μg LLP2A/mouse/100 μL) showed low bone marrow background signal as compared to mice imaged with a higher specific activity of ⁶⁴Cu-LLP2A (45.9 MBq/nmol; 0.08 μg LLP2A/mouse/100 μL). The standard uptake values (SUVₘₐₓ) in 5TGM-GFP subcutaneous tumors were calculated to be 5.53 vs 12.47 in the mice injected with a tracer of low and high specific activity respectively (FIG. 10). However, they demonstrated comparable tumor-to-muscle (42.50 vs 47.90) with both the molar activities. The background was high in mice injected with a specific activity of 45.9 MBq/nmol. Similarly, in mice (males and females) with disseminated myeloma disease, we noticed increased background with high specific activity radiotracer (FIG. 10). We observed that by using the low specific activity radiotracer, the background signal was reduced without losing the tumor signal. The radiotracer uptake in tumor-bearing sites such as the spine, femurs, and pelvis was comparable with both the molar activities. The data from the tissue biodistribution suggested no significant changes in the radiotracer uptake with both specific activities among healthy and tumor-bearing males. The healthy and tumor-bearing females, however, showed higher uptake in different organs like blood, bone, muscle, adrenal, spine, etc. when injected with a high specific activity of ⁶⁴Cu-LLP2A. In summary, a low specific activity product resulted in high-quality images, without compromising the specificity.

Proof-of-principle studies in a disseminated model of immunocompetent 5TGM1 mouse multiple myeloma using low specific activity tracer showed significant enhancements in the image quality and background uptake reduction in the bone marrow and spleen. This observation is substantial and counter-intuitive in the traditional sense. These novel in vivo findings demonstrated that a separate blocking dose may not be needed as done routinely with antibody imaging in humans. Indeed, with a slight adjustment in the specific activity, significant improvements in image quality could be achieved. Since LLP2A has a high affinity for the activated conformation of VLA4, we hypothesize that in tumor lesions that have a high density of VLA4 receptors, ligand-induced receptor clustering likely causes rapid tracer internalization followed by receptor saturation. Further mechanistic evaluations will help tease out the dynamics of the tracer uptake with varying specific activities.

### Example 3

To determine a new dose mass based on no-observed-adverse-effect level (NOAEL), i.e. no clinical signs of organ toxicity (vehicle vs experimental), the following experiments were conducted. The toxicity for the higher masses of the imaging agent, 90 μg and 60 μg were texted. A preliminary toxicology study was first performed using CD-1 IGS mice. Prior to conducting the toxicology study, the human equivalent dose (HED) was calculated based on the reference: Guidance for Industry: Estimating the Maximum Safe Starting Dose in Initial Clinical Trials for Therapeutics in Adult Healthy Volunteers. FDA, CDER guidelines, July 2005.

The calculation for one of the doses was performed using methods well-known in the art. According to FDA guidelines, HED (mg/kg) was estimated by dividing NOAEL (mg/kg) in each animal species by the appropriate body surface area conversion factor (BSA-CF). BSA-CF for mice is 12.3 and human weight was assumed to be 60 kg. When the planned mass limit is 90 μg per dose for a 60 kg human subject, the mass limit in mg/kg is 0.0015 mg/kg (90 μg/60 kg=1.5 μg/kg=0.0015 mg/kg). When setting HED for a 60 kg human subject to be X mg/kg with a safety factor of 150, and a planned mass limit of 90 μg per dose for a 60 kg human subject (0.0015 mg/kg), X is calculated as follows: X (mg/kg)/0.0015 (mg/kg)=150, thus X=150*0.0015 (mg/kg)=0.225 (mg/kg).

### Calculating Concentration of Cu-LLP2A Solution for Toxicology

HED for 60 kg human subject was 0.225 (mg/kg) and the animal dose was set as Y (mg/kg). Therefore, animal dose Y (mg/kg)/12.3=0.225 (mg/kg), thus Y=0.225 (mg/kg)*12.3=2.7675 (mg/kg). The assumed conditions for animal injections were determined as follows: concentration of Cu-LLP2A solution was Z mg/mL, injection volume for mice was 5 mL/kg, and average mouse weight was 25 g. The Cu-LLP2A amount injected into the mouse per kg was determined according to Z (mg/mL)*5 (mL/kg)=Z*5 (mg/kg). Injection volume per mouse was determined according to 5*25/1000=0.125 mL (per mouse). The concentration of Cu-LLP2A solution (Z μg/mL) was calculated as follows: Z(mg/mL)*5 (mL/kg)=2.7675 (mg/kg), thus Z (mg/mL)=2.7675 (mg/kg)/5 (mL/kg)=0.5535 (mg/mL). Similarly, the concentration for the 50 μg was determined.

### Toxicology Study

To evaluate the toxicity ^{[nat]}Cu-LLP2A, at two different dosages, when administered by single intravenous injection to CD1 IGS mice, the following experiments were conducted.

Once the concentrations were established, the solubility characteristics of the reference material ^{[nat]}Cu-LLP2A) were tested. Upon completing the initial QC, a preliminary toxicity study was performed. Both 90 μg and 50 μg concentrations were tested.

Sixty mice (30 male and 30 female) were initially randomly assigned to 12 groups of 5 animals/group. Two male groups and 2 female groups were given a single dose of normal saline (vehicle). Additionally, 2 male groups and 2 female groups were given single doses of ^{[nat]}Cu-LLP2A at 38.25 μg (1.54 mg/kg for 25 g mouse) The remaining 2 male groups and 2 female groups were given a single dose of ^{[nat]}Cu-LLP2A at 69.25 μg (2.77 mg/kg for 25 g mouse). The dose volume was 5 mL/kg (125 μL/mouse), and the dosing duration was less than 1 minute. Two days after administration, 1 male and 1 female group injected with the control vehicle, 1.54 mg/kg dose, or 2.77 mg/kg dose were sacrificed. Organs were observed and preserved. Blood samples were collected for clinical chemistry studies. Fourteen days after administration, 1 male and 1 female group each with a control vehicle, 1.54 mg/kg dose, or 2.77 mg/kg dose were sacrificed, and their organs were also observed and preserved. Blood samples were again collected for clinical chemistry studies. Criteria for evaluation included mortality, clinical observations, body weights, and blood chemistry.

No test article-related changes in body weights, no test article-related mortality at either dosage, and no physical signs of pain or distress were observed. No visible lesions were observed at necropsy, and no test article-related abnormal clinical signs were observed at either dosage. In conclusion, ^{[nat]}Cu-LLP2A, when administered once by intravenous injection at doses of 1.54 mg/kg or 2.77 mg/kg to CD-1 IGS mice, did not result in any mortality for the treated mice groups. Both dosages were well tolerated.

These data were collected for preliminary screening but may form the basis for more elaborate toxicology studies. Given no adverse effects were noted with either the 90 μg and 50 μg dose, a full-scale, exhaustive toxicity study with the 150×90 μg amount will be conducted.

### Generation of Calibration Curve for Accurate Determination of the Dose Concentration.

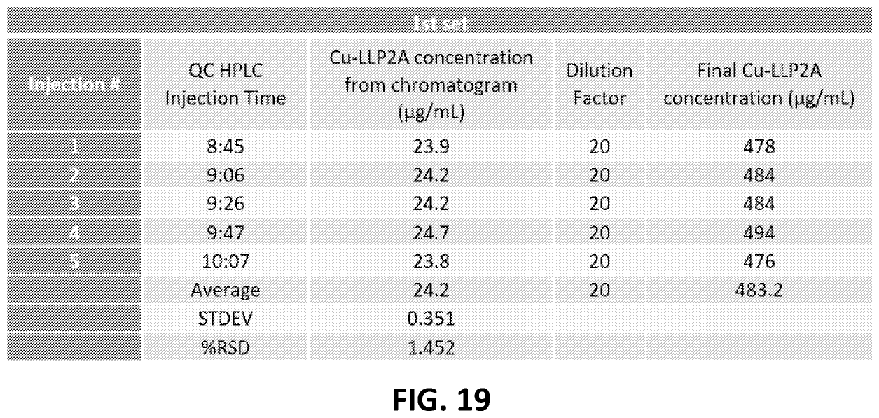

To accurately determine dosing, a calibration curve was generated using analytical HPLC. On the day of the toxicology study, a fresh sample was prepared and the concentration and stability were determined by sequential HPLC injections and analysis. A representative data set is summarized in FIG. 19.

After creating the calibration curve, the toxicology study was initiated. The dose was prepared per cyclotron-approved SOP. The sample was prepared under GMP conditions. The concentration and stability for the duration of the animal injections were determined via analytical HPLC (multiple injections for accuracy; see FIG. 19).

### Non-GLP Tolerability and Toxicity Study Following A Single Intravenous Administration of Cu-Labelled Peptidonminmetic, Cu-CB-TE1A1P-PEG4-LLP2A V2 (Cu-LLP2A V2), to Male and Female CD-1 Mice

To evaluate the tolerability and toxicity of Cu-CB-TE1A1P-PEG4-LLP2A V2 (Cu-LLP2A V2) tracer/imaging agent when administered once on Day 1 by intravenous (IV) bolus administration to male and female CD-1 mice, the following experiments were conducted. On Day 2 after administering the tracer/imaging agent, the first 10 animals/sex/group (Main Study) were sacrificed and necropsied. The remaining 5 animals/sex/group (Recovery) were weighed and observed for the remainder of the study and necropsied on Day 15.

Each treatment group (Groups 1-2) was comprised of 30 male and 30 female CD-1 mice. Mice were administered either vehicle or 0.0604 mg/mouse Cu-LLP2A V2 (Groups 1 and 2, respectively) once on Day 1 via IV bolus injection at a dose volume of 0.125 mL/mouse. Physical examinations were recorded at the time of randomization. Clinical observations were recorded daily. Body weight measurements were taken for randomization and daily. Blood samples for the evaluation of hematology and clinical chemistry were collected on Day 2 (Main Study) and Day 15 (Recovery). Following blood sample collections, a necropsy was conducted. Protocol-specified tissues were collected and evaluated grossly, select organs were weighed, and tissues were fixed for microscopic evaluation. Tissues were subsequently processed and evaluated microscopically.

All animals survived up until each animal's scheduled sacrifice. There were no abnormal observations or definitive Cu-LLP2A V2-related changes in body weights. No Cu-LLP2A V2-related findings in hematologic or clinical chemistry parameters were identified. There were no Cu-LLP2A V2-related changes in body weight, body/organ weight ratios, macroscopic findings, or microscopic findings in this study.

Based on the absence of abnormal clinical observations coupled with no definitive Cu-LLP2A V2-related changes in body weights, clinical pathology parameters, organ weights and associated ratios, and macroscopic/microscopic findings, a single IV injection of 0.0604 mg/mouse of Cu-LLP2A V2 was well tolerated in male and female CD-1 mice and was considered the NOEL (No Observable Effect Level) under the conditions of this study.

Based on the toxicity and tolerability data described above, the dosing ranges for three validation runs were determined: Specific Activity Range: 67-600 μCi/ug, Mass Range: 15-90 μg, and Activity/Patient Range: 6-13 mCi.

### Mean SUV Analysis of the Toxicology Study

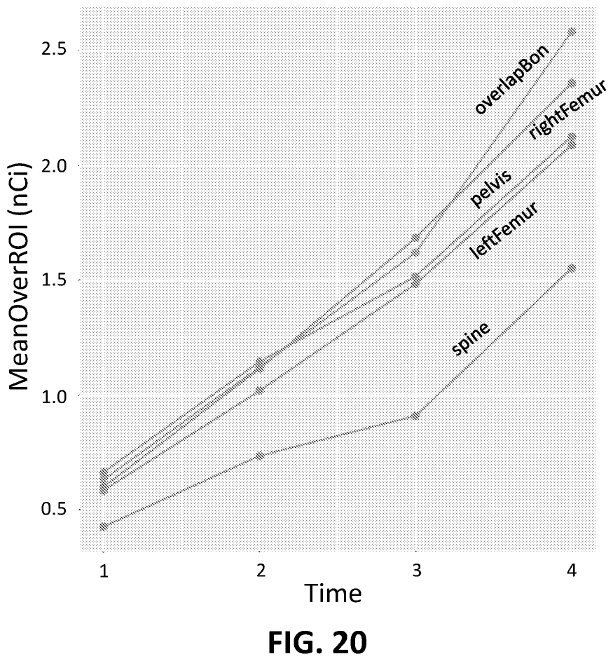

FIG. 20 is a graph summarizing the results of the Mean SUV Analysis of the toxicology study described above.

### Max SUV Analysis of the Toxicology Study

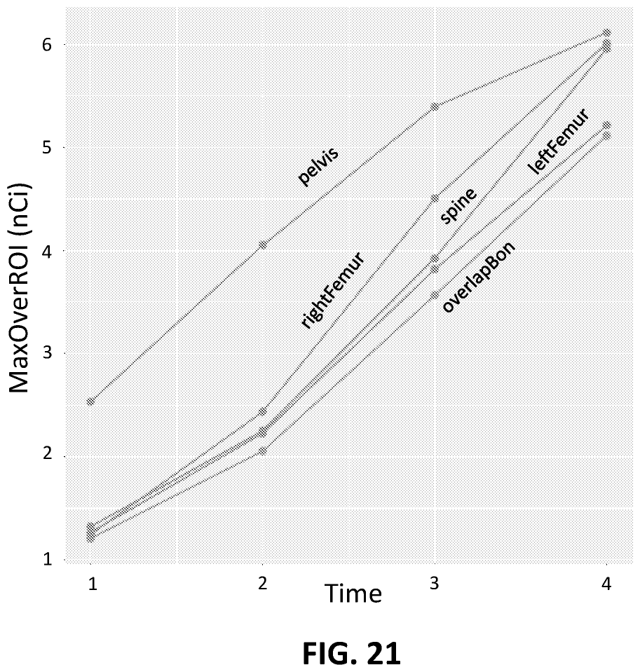

FIG. 21 is a graph summarizing the results of the Max SUV Analysis of the toxicology study described above.

1. An imaging agent composition, comprising a targeting ligand covalently attached to or complexed with a radionuclide binding structure, the targeting ligand configured to preferentially bind activated VLA4, and the radionuclide binding structure configured to bind or complex to a radionuclide.

2. The composition of claim 1, wherein the targeting ligand is LLP2A and modifications thereof.

3. The composition of claim 1, wherein the radionuclide binding structure comprises a chelator.

4. The composition of claim 3, wherein the chelator comprises one of 1,4,8,11-tetraazacyclotetradecane-1-(methane phosphonic acid)-8-(methane carboxylic acid) (TE1A1P), dodecane tetraacetic acid (DOTA), diethylenetriaminepentaacetic acid (DTPA), and 1,4,8,11-tetraazacyclotetradecane-1-(methane phosphonic acid)-8-(methane carboxylic acid) (CB-TE1A1P).

5. The composition of claim 1, wherein the imaging agent comprises one of LLP2A-11-bis(carboxymethyl)-1,4,8,11-tetraazabicyclo[6.6.2]hexadecane (LLP2A-CB-TE2A), LLP2A-1,4,7,10-tetraazacyclododecane-N,N′,N″,N′″-tetraacetic acid (LLP2A-DOTA), and LLP2A-DOTA-polyethylene glycol (LLP2A-DOTA-PEG).

6. The composition of claim 1, wherein the radionuclide binding structure is 1,4,8,11-tetraazacyclotetradecane-1-(methane phosphonic acid)-8-(methane carboxylic acid) (CB-TE1A1P).

7. The composition of claim 1, wherein the radionuclide comprises a radiometal.

8. The composition of claim 7, wherein the radiometal comprises one of ⁶⁴Cu, ¹¹¹In, and ⁶⁸Ga.

9. The composition of claim 8, wherein the radiometal is ⁶⁴Cu.

10. The composition of claim 1, wherein the imaging agent is [⁶⁴Cu]Cu-LLP2A.

11. The composition of claim 1, further comprising a linker covalently attached to or complexed with the targeting ligand and the radionuclide binding structure, wherein the linker is configured to attach the targeting ligand to the radionuclide binding structure.

12. The composition of claim 11, wherein the targeting ligand, the radionuclide binding structure, the linker, and any combination thereof are configured to modulate the activation of VLA4.

13. The composition of claim 1, wherein a clinical dose of the composition comprises:
a. from about 15 μg to about 100 μg of a non-radioactive peptide portion comprising the targeting ligand, the radionuclide binding structure, the linker, and any combination thereof; and
b. from about 7 mCi to about 73 mCi of ⁶⁴Cu.

14. (canceled)

15. (canceled)

16. A method for imaging malignant bone marrow in a subject, the method comprising:
a. administering a clinical dose of the imaging agent composition of claim 1;
b. performing PET imaging on the subject; and
c. spatially identifying lesions in the bone marrow based on the PET images.

17. The method of claim 16, wherein the PET imaging is performed from about one hour to about 6 hours after administering the clinical dose of the imaging agent composition.

18. (canceled)

19. A method for monitoring for relapse in malignant bone marrow in a subject, the method comprising:
a. obtaining a baseline spatial map of lesions in the bone marrow of the subject after completion of a treatment and at least one follow-up map of lesions in the bone marrow of the subject at least once after obtaining the baseline spatial map, wherein each spatial map is obtained by:
i. administering a clinical dose of the imaging agent composition of claim 1;
ii. performing PET imaging on the subject; and
iii. transforming the PET image into the spatial map of the lesions in the bone marrow;
b. comparing the at least one follow-up map to the at least one baseline spatial map; and
c. identifying a relapse if any of the at least follow-up maps comprise bone marrow lesions more extensive than the baseline map.

20. The method of claim 19, wherein the PET imaging is performed from about one hour to about 6 hours after administering the clinical dose of the imaging agent composition.

21. (canceled)

22. A method for selecting a treatment for malignant bone marrow in a subject, the method comprising:
a. administering a clinical dose of the imaging agent composition of claim 1;
b. performing PET imaging on the subject; and
c. transforming the PET image into a spatial map of the lesions in the bone marrow; and
d. selecting a treatment based on the spatial map of the lesions in the bone marrow.

23. The method of claim 22, wherein the PET imaging is performed from about one hour to about 6 hours after administering the clinical dose of the imaging agent composition.

24. (canceled)

25. The method of claim 16, wherein the malignant bone marrow is associated with multiple myeloma.
